(19) United States
(12) Patent Application Publication
(10) Pub. No.: US 2025/0194987 A1
(43) Pub. Date: Jun. 19, 2025

(54) SYSTEM AND A METHOD FOR DETERMINING A HEALTH CONDITION OF A SUBJECT

(71) Applicant: The Chinese University of Hong Kong, Hong Kong (CN)

(72) Inventor: Kam Lung Kelvin CHONG, Hong Kong (CN)

(21) Appl. No.: 18/976,842

(22) Filed: Dec. 11, 2024

Related U.S. Application Data
(60) Provisional application No. 63/611,955, filed on Dec. 19, 2023.

(51) Int. Cl. A61B 5/00; A61B 3/00; A61B 3/08; A61B 3/11; A61B 3/113; A61B 3/14

(52) U.S. Cl. CPC A61B 5/4076; A61B 3/005; A61B 3/0075; A61B 3/08; A61B 3/112; A61B 3/113; A61B 5/7267; A61B 3/14

(57) ABSTRACT

A system and a method for determining a health condition of a subject. The system comprises: a display module arranged to provide visual stimulus to the subject; an image capturing module arranged to record a binocular response performed by the subject in response to the visual stimulus being received; and a processor module arranged to analyze a plurality of parameters extracted from the recorded binocular response to determine the neurological status and/or neurological activity of the subject, thereby determining one or more symptoms associated with the health condition of the subject.

### TECHNICAL FIELD

This invention relates to a system and a method for determining a health condition of a subject. Particularly, although not exclusively, the invention relates to a non-intrusive system and method for determining neurological manifestations based on pupillometry analysis.

### BACKGROUND OF THE INVENTION

One of the challenges in detecting long-COVID is identifying its neurological signs, which can be subtle and difficult to diagnose. However, studies have shown an increased risk of neurological disorders, such as stroke, cognition and memory disorders, and autonomic nervous system complications in individuals after being infected with SARS-CoV-2.

On the other hand, psychoactive substance abuse (PSA) has been a long-standing issue causing harm to physical, psychological and social wellbeing. There are more than 5 thousand reported drug users in Hong Kong every year. Although the trend has decreased in recent years, illicit drug use, especially among the youth, is a public health issue warranting greater attention in Hong Kong. Heroin, methylamphetamine, cocaine, cannabis and ketamine are the five leading substances abused in Hong Kong since 2018. A recent governmental report demonstrated heroin and methylamphetamine were among the most common psychoactive drugs affecting 40.0% and 18.7% of previously reported drug abusers in the first-to-third quarters of 2021. Furthermore, approximately 60% and 45% of abusers who aged under 21 were cannabis and cocaine users respectively, with a raising trend in cannabis use among the youth.

### SUMMARY OF THE INVENTION

In accordance with a first aspect of the present invention, there is provided a method for determining a health condition of a subject, comprising the steps of: providing visual stimulus to the subject; recording a binocular response performed by the subject in response to the visual stimulus being received; and analyzing a plurality of parameters extracted from the recorded binocular response to determine the neurological status and/or neurological activity of the subject, thereby determining one or more symptoms associated with the health condition of the subject.

In accordance with the first aspect, the binocular response includes binocular pupillometry response and reactivity.

In accordance with the first aspect, the plurality of parameters includes at least one of maximum pupil diameter, minimum pupil diameter, absolute construction amplitude, constriction velocity, constriction latency, construction time, redilation velocity and post-illumination pupil response amplitude, relative amplitude, ⅓ redilation time, percentage of pupil constriction, recovery time to achieve 75% of maximal pupil size (T75) and ⅔ constriction time, latency, response time, rapid phase dilation velocity and slow phase dilation velocity.

In accordance with the first aspect, the health condition includes disease or disorder caused by neurodevelopmental, neurodegenerative, neuropsychiatric, neurocognitive or neurobehavioral issues.

In accordance with the first aspect, the health condition includes long-COVID, post-COVID or psychoactive drug abuse.

In accordance with the first aspect, the binocular response further includes tracked eye movements in response to the visual stimulus.

In accordance with the first aspect, the visual stimulus is provided to the subject according to a stimulus protocol within a predetermined period of time.

In accordance with the first aspect, the method comprises the step of generating pupillary light reflex (PLR) data for each eye synchronized with the stimulus protocol based on the recorded binocular response, wherein the plurality of parameters are extracted from the pupillary light reflex data.

In accordance with the first aspect, the visual stimulus is provided to the subject using a VR head-mount display apparatus.

In accordance with a second aspect of the present invention, there is provided a system for determining a health condition of a subject, comprising: a display module arranged to provide visual stimulus to the subject; an image capturing module arranged to record a binocular response performed by the subject in response to the visual stimulus being received; and a processor module arranged to analyze a plurality of parameters extracted from the recorded binocular response to determine the neurological status and/or neurological activity of the subject, thereby determining one or more symptoms associated with the health condition of the subject.

In accordance with the second aspect, the binocular response includes binocular pupillometry response and reactivity.

In accordance with the second aspect, the plurality of parameters includes at least one of maximum pupil diameter, minimum pupil diameter, absolute construction amplitude, constriction velocity, constriction latency, construction time, redilation velocity and post-illumination pupil response amplitude, relative amplitude, ⅓ redilation time, percentage of pupil constriction, recovery time to achieve 75% of maximal pupil size (T75) and ⅔ constriction time, latency, response time, rapid phase dilation velocity and slow phase dilation velocity.

In accordance with the second aspect, the health condition includes disease or disorder caused by neurodevelopmental, neurodegenerative, neuropsychiatric, neurocognitive or neurobehavioral issues.

In accordance with the second aspect, the health condition includes long-COVID or post-COVID or psychoactive drug abuse

In accordance with the second aspect, the binocular response further includes eye movements in response to the visual stimulus.

In accordance with the second aspect, the display module is arranged to provide visual stimulus associated with a stimulus protocol within a predetermined period of time.

In accordance with the second aspect, the stimulus protocol is programmable, and wherein the stimulus protocol includes attributes including at least one of light intensity, duration, interval, wavelength, specific video stimuli and displayed pattern/images.

In accordance with the second aspect, the processor module is further arranged to generate pupillary light reflex (PLR) data synchronized with the stimulus protocol for each eye based on the recorded binocular response, wherein the plurality of parameters are extracted from the pupillary light reflex data.

In accordance with the second aspect, the processor module includes a machine learning processing network.

In accordance with the second aspect, the display module includes a VR head-mount display apparatus.

In accordance with the second aspect, the VR head-mount display apparatus comprises an eye spacing adjustment mechanism for adjusting a distance between a display of the VR head-mount display apparatus and eyes of the subject.

In accordance with the second aspect, the image capturing module includes a pair of eye-tracking video cameras arranged to capture the binocular response.

### BRIEF DESCRIPTION OF THE DRAWINGS

Embodiments of the invention will now be described, by way of example, with reference to the accompanying drawings in which:

FIG. 1 is a block diagram illustrating a system for determining a health condition of a subject in accordance with an embodiment of the present invention.

FIG. 2 is an illustration of the infrared pupillometry task with three stages of different light intensities and dark stages.

FIG. 3 is a Normal Schematic Diagram with Pupillary Light Reflex Indices.

FIG. 4 is a screen shot showing calibration during VR iP&ET test.

FIG. 5 are plots illustrating normal pupillary response in both eyes which shows steady pupil constriction and smooth pupil dilation.

FIG. 6 are plots illustrating normal pupillary response in an alternative example.

FIG. 7 are plots showing pupillary unrest in pupillometry tracing of both eyes from a poly-drug subject with 15 years history of PSA.

FIG. 8 are plots showing pupillary unrest in pupillometry tracing of both eyes from a poly-drug subject with 15 years history of PSA in an alternative example.

FIG. 9A is a chart showing comparison of relative amplitude between the psychoactive substance abusers (blue) and the health controls (orange).

FIG. 9B is a chart showing comparison of ⅓ redilation time between the psychoactive substance abusers (blue) and the health controls (orange).

FIG. 9C is a chart showing comparison of percentage of pupil constriction between the psychoactive substance abusers (blue) and the health controls (orange).

FIG. 9D is a chart showing comparison of recovery time to achieve 75% of maximal pupil size (T75) between the psychoactive substance abusers (blue) and the health controls (orange).

FIG. 9E is a chart showing comparison of % constriction time between the psychoactive substance abusers (blue) and the health controls (orange).

FIG. 10 are plots showing pupillary light reflex in a healthy subject.

FIG. 11 are plots showing pupillary light reflex in a psychoactive substance abuser.

FIG. 12 are plots showing pupillary light reflex in a former psychoactive substance abuser.

FIG. 13 is a plot showing AUC of ⅓ redilation time in PLR 128.

FIG. 14A is a plot showing AUC of relative amplitude in PLR 255.

FIG. 14B is a plot showing AUC of percentage of pupil constriction in PLR 255.

FIG. 14C is a plot showing AUC of ⅓ redilation time in PLR 255.

FIG. 15 is a plot showing AUC of the CNN-LSTM in distinguishing 10 pupillometric parameters of the PSA and HC groups.

FIG. 16 is a plot showing AUC of the CNN-LSTM in distinguishing the pupillary unrest of the PSA and HC groups.

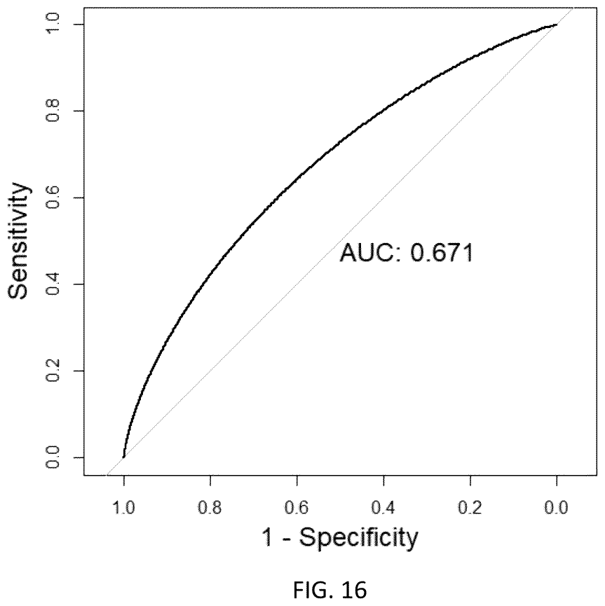

FIG. 17 is a block diagram illustrating a workflow of analysis of PLR data obtained for distinguishing subjects of long-COVID/post-COVID and HC groups.

### DETAILED DISCLOSURE OF THE INVENTION

The inventors, through their experiments and trials, devised that Pupillary light reaction (PLR) is known to subject to the effects of psychoactive substances (PAS) through the autonomic nervous system. Stimulants such as crystal methamphetamine (Ice), ketamine, and cocaine cause pupil dilatation (mydriasis) while narcotics including heroin cause pupil constriction (miosis). Although definition of a “normal” pupil size is currently lacking due to inter-individual variation of pupil size secondary to the effect of age, emotion, fatigue, medical and ocular condition as well as systemic and ocular medications, the above PAS were shown to impair PLR which in turn can be quantified by infrared pupillometry (iP) as the temporal sequence of changes in the size of the pupil upon light stimulus. Basic components in iP include response time, ⅔ constriction time, ⅓ redilatation time and slow-phase dilatation velocity using light of low, medium, and highest intensities (PLR 64, 128, 255) or that based on initial pupil diameter (PLR+).

Virtual reality (VR) has been increasingly implemented in various aspects of clinical application, including education and training tools for medicine, exposure therapy for phobias, treatment for addictive disorders and neurorehabilitation. VR excels in the realism of the images and provides a three-dimensional perception to users.

To measure the drug-related pupil response by VR-based infrared pupillometry, the inventors conducted a study which aimed to construct an age-, sex-, ethnic-specific, normative and substance-related local databases on VR-based IP parameters as well as to investigate the prevalence of eye-related symptoms and visual harm among drug abusers.

Preferably, high-speed iP may be measured within a virtual-reality (VR) headset. Advantageously, it provides a portable, enclosed and standardized testing environment without ambient light disturbance which is crucial for iP. It can also track eye movements during display of pictures or videos designed for further evaluation or education. In addition, eye-tracking (ET) may detect cognitive components of PAS use, such as attentional biases, which are related to drug-seeking behaviour and relapse during rehabilitation. Attentional bias for PSA means their subconscious tendency to focus on or pay attention to PAS-related cues such as crystals, pills, powder, and paraphernalia. Attentional bias may explain why PSA seem to be surrounded by relatively many temptations, which makes abstinence even more challenging. Recent research supports the idea that attentional bias towards drug cues is related to the intensity and persistence of the addictive behaviours. Dependent subjects were found to exhibit greater anti-saccade errors, longer reaction time latencies, and greater pupil diameters (during fixation) on drug-related stimuli. Saccade (eye movement)-based measurement of attentional bias was found useful to assess reactivity to drug cues and to screen for potential relapse prevention interventions.

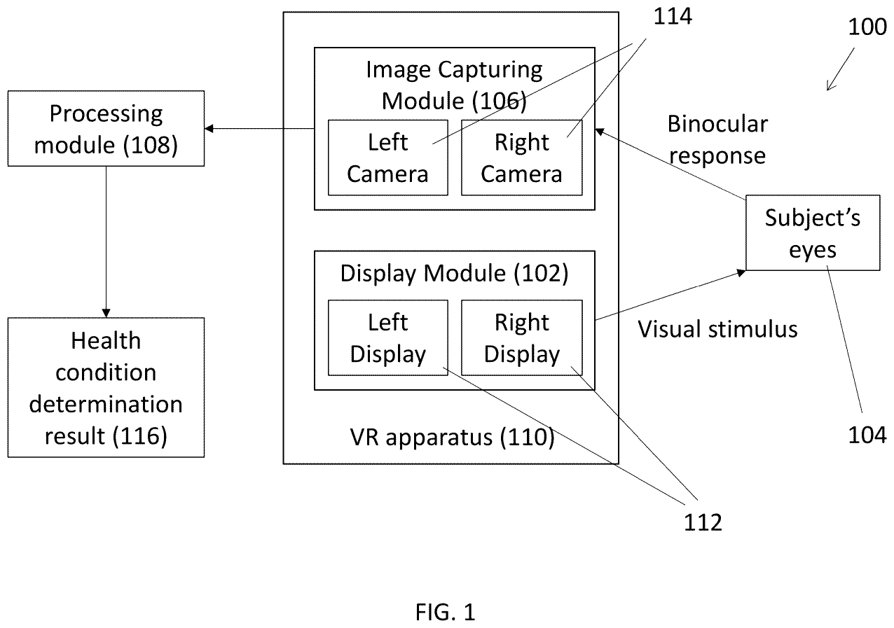

With reference to FIG. 1, there is shown an example embodiment of a system 100 for determining a health condition of a subject, comprising: a display module 102 arranged to provide visual stimulus to the subject 104; an image capturing module 106 arranged to record a binocular response performed by the subject 104 in response to the visual stimulus being received; and a processor module 108 arranged to analyze a plurality of parameters extracted from the recorded binocular response to determine the neurological status and/or neurological activity of the subject 104, thereby determining one or more symptoms associated with the health condition of the subject 104.

In this embodiment, the system comprises two main parts—a head-mount unit to be “worn” by a subject or patient and a processing module for analysing data collected by the head-mount unit. Preferably, the head-mount unit may be a virtual reality (VR) display apparatus 110 having two display units 112 each for providing visual stimulus to left or right eye of the subject. For example, VR content such as 3D image or video, as a form of visual stimulus, may be project to the user eye. Alternatively, other forms of visual stimulus, such as color pattern, or simply light wave of predetermine wavelength/color, may also be used in some example embodiments.

Referring to FIG. 1, the VR display apparatus 110 further comprises an image capturing module, i.e. two cameras 114, for record a binocular response performed by the subject 104 in response to the visual stimulus provided by the display module 102. Preferably, the binocular response includes binocular pupillometry response and reactivity. Advantageously, by capturing and analyzing data from both eyes simultaneously, a more comprehensive understanding of a subject's pupil reactivity and response can be obtained. This enables more accurate and robust assessments of various conditions.

Preferably, the image capturing module 106 may include a pair of eye-tracking video cameras arranged to capture the binocular response. For example, a binocular eye-tracking camera add-on specifically designed for tracking parameters of eye movement may be used to record real time eye-tracking images/video of the eyes of the subject when VR content is displayed to the subject. A part from recording the movement of eyeballs, pupils of the subject's eyes can also be recorded simultaneously to enable analysis of pupillometry response and reactivity, and the pupillometry parameters may further assist determination of the neurological status and/or neurological activity, thus a health condition, of the subject.

Preferably, parameters extracted from the recorded binocular response may include maximum pupil diameter, minimum pupil diameter, absolute construction amplitude, constriction velocity, constriction latency, construction time, redilation velocity and post-illumination pupil response amplitude, relative amplitude, ⅓ redilation time, percentage of pupil constriction, recovery time to achieve 75% of maximal pupil size (T75) and ⅔ constriction time, latency, response time, rapid phase dilation velocity and slow phase dilation velocity.

In addition, the binocular response further includes eye movements in response to the visual stimulus. For example, the system may also analyze the subject's psychological behavior when contextual information is provided as visual stimulus to the subject. In contrast to other approaches that solely focus on capturing one single pupillometry tracing after one single stimulus, this invention goes beyond and can automatically program multiple stimuli and capture the corresponding responses continuously, and binocular response may be captured simultaneously.

In one exemplary embodiment, the VR display apparatus may be a HTC cosmos head mounted display with Droolon F1® eye-tracking add-on included as the image capturing module. HTC Cosmos head mounted display (HMD) is integrated with eye-tracking Droolon F1® add-on. HMD with dual 3.4″ diagonal screen ad 1440×1700 pixels per eye (2880×1700 pixels combined) and 90 hz refresh rate and 110 degrees field of view. In an alternative experiment, the apparatus may be a HTC Vive Pro Eye with Pupil Lab eye-tracking add-on included as the image capturing module.

In addition, the system 100 further comprises a processor module 108 for analyzing the recorded binocular responses, so as to output results of determination of health condition 116. More preferably, the processor module 108 is a machine learning processing network for providing a comprehensive analysis of the binocular response to identify neurological manifestations based on the binocular response captured by the VR display apparatus.

With the abovementioned parameters, the processing module 108 is able to capture, identify, and characterize pupillometry tracings. These tracings provide valuable information on neurological status and activity by analysing the pupil reactivity and response, which facilitate classifying individuals into different types, namely Healthy Control (HC), Post COVID (PCVD), and Long COVID (LCVD).

The parameters are useful in detection, classification, and monitoring of individuals with long-COVID and Post COVID. By comparing the specific segment and parameters, such as Baseline Pupil Diameter, Minimum Diameter, Absolute Construction Amplitude, Constriction Velocity, Constriction Latency, Construction Time, Redilation Velocity, and post-illumination pupil response (PIPR) Amplitude, with a corresponding control parameter, differences may be assessed and identified. This enables healthcare professionals to monitor and provide appropriate medical interventions and treatment plans for individuals experiencing prolonged effects or complications from COVID-19. Additionally, this system and method can contribute to a better understanding of Long-COVID/Post COVID and its neurological manifestations, leading to improved management and care for affected individuals.

For the respective of detecting psychoactive substance abuse (PSA), the processing module may identify, and characterize pupillometry tracings, which selectively capture and compare key pupillometry parameters for PSA detection.

There are significant difference of pupil reactivity and response in some segment of whole pupillometry between psychoactive substance abuse and healthy control. By comparing the specific parameters, such as relative amplitude, % redilation time, percentage of pupil constriction, recovery time to achieve 75% of maximal pupil size (T75) and % constriction time between, with a corresponding control parameter, the system can accurately classify individuals into psychoactive substance abuse and control.

Advantageously, embodiments of the invention may aid law enforcement agencies, drug rehabilitation centers, schools, and other relevant organizations in the early detection, classification and monitor of individuals with Long-COVID and Post-COVID, psychoactive substance abuse. By offering a non-intrusive solution that can effectively capture and analyze pupillometry tracings, while the pupillometry tracings will be analyzed and interpretated as the windows of brain and provide insight of neurological activities.

In addition, the invention also provides a significant advancement, where it is an affordable, repeatable, non-intrusive, in the field of Long-COVID, drug abuse detection which support remote and self-monitoring.

In a preferred embodiment, a combination of Convolutional Neural Network (CNN) and Long Short-term Memory (LSTM) was employed in the machine learning processing network of the processing module 108 to analyze the pattern of pupillary unrest and the comparison between the PSA and HC groups. A CNN is a type of artificial neural network that contains an input layer, multiple hidden layers, and an output layer. The hidden layers perform convolutions that formulate a feature map that would contribute to a subsequent layer. A LSTM is a recurrent neural network that shares both “long-term memory” and “short-term memory”. It is consisted of a cell, an input gate, an output gate and a forget gate where feedback connections could be achieved. In a preferred embodiment, two deep learning process (CNN-LSTM) were combined. The machine learning network includes altogether two convolutional layers, one max-pooling layer, one flatten layer, two LSTM layers, and one dense layer. The total number of timesteps may be 558.

The machine-learning processing network may be implemented as a processor within a computer server or the external computer which process the sampled data. In this embodiment, the system comprises a server which includes suitable components necessary to receive, store and execute appropriate computer instructions. The components may include a processing unit, including Central Processing Unit (CPUs), Math Co-Processing Unit (Math Processor), Graphic Processing Unit (GPUs) or Tensor processing united (TPUs) for tensor or multi-dimensional array calculations or manipulation operations, read-only memory (ROM), random access memory (RAM), and input/output devices such as disk drives, input devices such as an Ethernet port, a USB port, etc. Display such as a liquid crystal display, a light emitting display, or any other suitable display and communications links. The server may include instructions that may be included in ROM, RAM or disk drives and may be executed by the processing unit. There may be provided a plurality of communication links which may variously connect to one or more computing devices such as a server, personal computers, terminals, wireless or handheld computing devices, Internet of Things (IoT) devices, smart devices, edge computing devices, cloud devices. At least one of a plurality of communications links may be connected to an external computing network through a telephone line or other type of communications link.

The server may include storage devices such as a disk drive which may encompass solid state drives, hard disk drives, optical drives, magnetic tape drives or remote or cloud-based storage devices. The server may use a single disk drive or multiple disk drives, or a remote storage service. The server may also have a suitable operating system which resides on the disk drive or in the ROM of the server.

The computer or computing apparatus may also provide the necessary computational capabilities to operate or to interface with a machine learning network, such as neural networks, to provide various functions and outputs. The neural network may be implemented locally, or it may also be accessible or partially accessible via a server or cloud-based service. The machine learning network may also be untrained, partially trained or fully trained, and/or may also be retrained, adapted or updated over time.

In a preferred embodiment, the display module 102 may provide visual stimulus associated with a stimulus protocol within a predetermined period of time. During a session, after wearing a VR-HMD properly, the subject may be shown three different intensities of light stimuli, where the protocol of cycles of light stage.

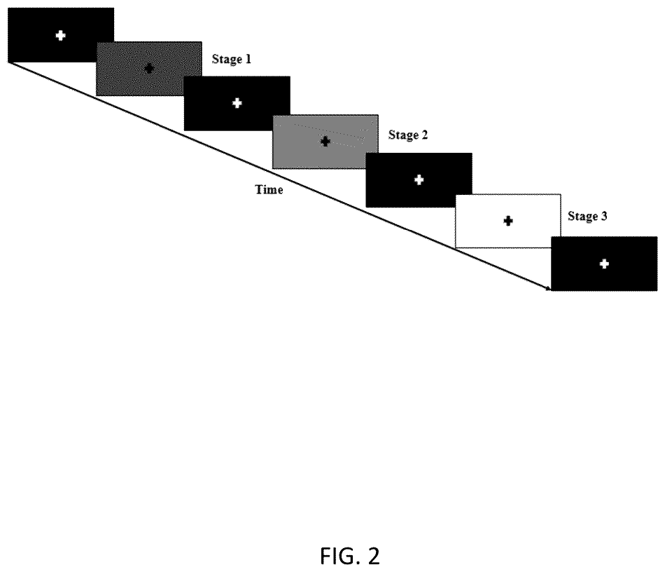

Referring to FIG. 2, there is shown an example stimulus protocol, for measuring pupillary light reflex (PLR) by alternating total 3 stages of dark and light. In this example, a 13-second dark stage would be firstly initiated for pupil constriction, followed by a 2-second light stage for pupil redilatation. Such cycle was conducted consecutively for 3 times with incremental light intensity by the VR headset with pupil dynamics captured by IP. For every dark stage, the light intensity was PLR 0 (RGB 0, 0, 0). For the first, second and third light stage, the light intensities were PLR 64 (RGB 64, 64, 64), PLR 128 (RGB 128, 128, 128) and PLR 255 (RGB 255, 255, 255).

Preferably, the stimulus protocol is programmable, and wherein the stimulus protocol includes attributes including at least one of light intensity, duration, interval, wavelength, specific video stimuli and displayed pattern/images. For example, visual stimulus of light with different color and wave length, showing different pattern, video with different latency, time and frequency may be provided to the subject.

Instead of just using white light as stimulus which is not modifiable, using an upgradable virtual reality head-mount display platform may provide visual stimulus of different light intensities, duration, wavelengths, patterns and even videos as stimuli per operators/users' requirement. Such flexibility for stimuli and immersive experience for users allow the detection of various unrelated conditions with a much deeper level of analysis to enhance the precision. As new detection threshold or diagnostic criteria emerge, the system can be updated without significant hardware modification.

Optionally, in order to facilitate more accurate data collection, the subjects may be asked to reduce the head movement during the task, and in order to measure an accurate mapping of eye movement, nine-point calibration was performed before the IP test.

In addition, the VR head-mount display apparatus 110 may further comprise an eye spacing adjustment mechanism for adjusting a distance between a display of the VR head-mount display apparatus and eyes of the subject, which allows the subject to adjust the optimal distance for viewing the displayed contents provided by the VR head-mount display.

In addition, VR prescription lens inserts that can magnetically attach to the lens of the VR display apparatus may be provided, which allow subjects to take off their glasses and prevent optical interference during VR iP&ET test.

Preferably, the processor module is further arranged to generate pupillary light reflex (PLR) data synchronized with the stimulus protocol for each eye based on the recorded binocular response, wherein the plurality of parameters are extracted from the pupillary light reflex data.

When visual stimulus is displayed to the subject's eyes, subjects' pupil size will be captured with high speed sampling rate via infrared pupillometry in VRHMD. The subjects' pupillary data is then collected by micro-cameras inside the VR-HMD, and the software generates graphs in real-time. The pupil size of the participants changes under three different light intensity. The system collected real-time binoculars pupil size of participants, and generate two Pupillary Light Reflex (PLR) for analysis.

Without wishing to be bound by theory, there are 4 dynamics phases of PLR, response latency, maximum constriction, pupil escape, and recovery are included in PLR follow. PLR consists of 2 processes, light stimulus on and light stimulus off, which is also called post-illumination pupil response (PIPR).

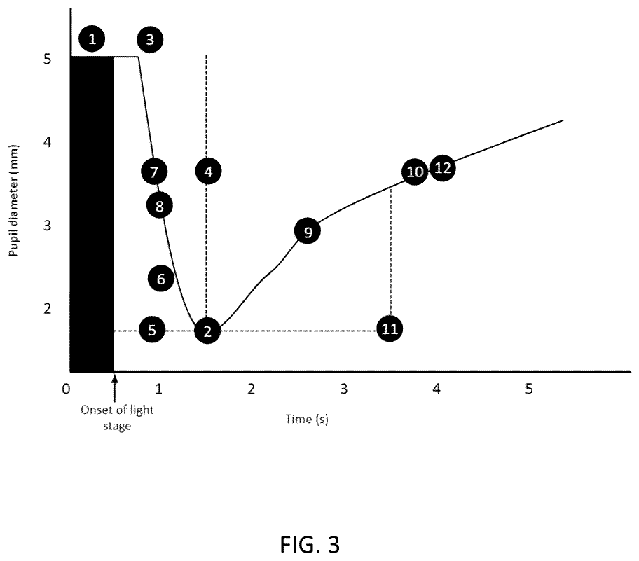

With reference to FIG. 3, there is shown the pupillary response during the light stage. All pupillary metrics were calculated with the following definition. Maximum pupil diameter was measured in the dark stage. Minimum pupil diameter was measured in the light stage. Latency was defined as the time between the light stage initiation and the onset of pupillary reaction. Relative amplitude was calculated as the ratio between amplitude of pupillary reaction and pupil diameter on light stage initiation. Response time was calculated by the time between the light stage initiation and the time when minimum pupil diameter was reached. % constriction time was defined as the time between the onset of pupil response and the time when % of the reflex amplitude was reached. Average constriction velocity was calculated from the average velocity of the constriction of the pupil during a linear phase of pupil constriction, which was set between 40 and 80% of the amplitude. Percentage of pupil constriction was defined as the percentage of the time between the onset of pupil response and the time when the reflex amplitude was reached. Dilation velocity (rapid phase) was defined as the velocity of dilation at the beginning of the dilation phase (set between 20 and 35% of the amplitude). Dilation velocity (slow phase) was defined as the velocity of dilation at the end of the dilation phase (set between 80 and 95% of the amplitude). ⅓ redilation time was determined by the time when minimal pupil diameter was reached and the time when ⅓ of reflex amplitude was achieved. 75% recovery time represented the time to reach 75% recovery of the reflex amplitude from the minimal pupil diameter.

In this example, total 12 pupillary metrics were evaluated pupillary light reflex by virtual reality-based in three light stages: 1) maximum pupil diameters; 2) minimum pupil diameters; 3) latency; 4) relative amplitude; 5) response time; 6) % constriction time; 7) average constriction velocity; 8) percentage of pupil constriction; 9) dilation velocity (rapid phase); 10) dilation velocity (slow phase); 11) ⅓ redilation time; and 12) recovery time (T75).

In experiments performed by the inventors, infrared pupillometry was first performed using the VR headset with the Droolon F1® eye-tracking add-on cameras, which provides latency less than 5 ms and 120 hz sampling rate. Measurements will be taken on each eye and results of both eyes will be selected for use. Initial pupil size, response time, ⅔ constriction time, ⅓ re-dilatation time and slowphase dilatation velocity using light of low (PLR 64) (RGB 64, 64, 64), medium (PLR 128) (RGB 128, 128, 128) and highest intensities (PLR 64) (RGB 255, 255,255) that based on initial pupil diameter (PLR+) will be recorded.

Repeatability and reproducibility were evaluated based on the definitions adopted by the British Standards Institution. Under repeatability conditions, independent test results are obtained with the same method, on the same subject, by the same operator, and on the same set of equipment with the shortest time lapse possible between successive sets of readings. Repeatability was investigated by completing 10 sets of iP&ET tests. All scanning will be performed by the same operator. The time elapsed between successive test will correspond to the time taken to save the previous data and the headset will be adjusted if necessary for not more than a few seconds. Repeatability will be evaluated with 5 urinenegative healthy volunteers. Inter-session reproducibility will be examined with another 5 urinenegative healthy volunteer 2 weeks apart for three sets of iP&ET tests.

Eye-tracking technology may also be adopted to measure performance on counterbalanced blocks of pro-saccade and anti-saccade trials featuring drug-related and neutral stimuli (pictures). Dependent measurements including anti-saccade errors, saccadic response times and latencies, and pupil diameter during fixation on stimuli will be recorded.

Preferably, symptoms that are caused by the subject's neurological status and/or neurological activity may be detected by the system 100 in accordance with embodiments of the present invention. For example, the health condition includes long-COVID or post-COVID, which cause neurocognitive issues, or psychoactive drug abuse (PSA) which causes neuropsychiatric issues.

For the respective of detecting long-COVID and post-COVID conditions, the above features from PLR curve are extracted, machine learning models with unique data processing method are also used to identify potential ocular biomarkers for predicting disease status.

For the respective of detecting psychoactive drugs abusers, segments from PLR curve are extracted, machine learning models with unique data processing method are also used to classify individuals into psychoactive substance abuse and non-psychoactive substance abuse categories.

Preferably, the system may be modified or adapted for identifying health conditions including other disease or disorder caused by neurodevelopmental, neurodegenerative, neuropsychiatric, neurocognitive or neurobehavioral issues. Examples of these health conditions includes:
Neurodevelopmental: e.g. pediatric, ADHD, ASD;
Neurodegenerative: e.g. geriatric, PD, dementia/MCI;
Neuropsychiatric: e.g. PSA, depression, psychosis, anxiety, substance abuse;
Neurocognitive: e.g. Long Covid, concentration, focus, mindfulness; and
Neurobehavioral: e.g. sports, surgical training.

These health conditions involve changes or abnormalities in brain function or structure. This can include how the brain develops, how it processes information, or how it controls behavior and emotions. It may be advantageous to identify whether a subject may have any of these health conditions as these diseases or disorders may impact an individual's daily life, affecting cognitive abilities, behavior, emotions, and overall mental health.

It should be appreciated by a skilled person in the art that the system may be modified or adapted for detecting symptoms associated with one or more of the abovementioned health conditions, e.g. by training the machine learning processing network with training dataset associated with the corresponding health condition.

Inventors further conducted exemplary experiments to evaluate the performance of embodiments of the present invention. Example 1 and 2 relates to experiments conducted for identifying subjects affected by PSA, and Example 3 relates to an experiment conducted for identifying subjects affected by long COVID or post COVID, after modifying the system 100 for determining symptoms caused by neuropsychiatric issues to neurocognitive issues.

### Example 1

A total of 625 subjects participated in iP&ET while 606 completed all study procedures, they were divided into 4 groups.
1. Group 1: 300 secondary school students participating in the Healthy School Programme with a Drug Testing Component
2. Group 2: 100 students from tertiary institutes
3. Group 3: 100 high-risk youth and young adults outside school network who are clients of the collaborating non-governmental organizations
4. PSA group: 106 rehabilitees recruited from Counselling Centres for Psychotropic Substance Abusers (CCPSA), drug treatment and rehabilitation centres (DTRC) and clinics attended by PSA with 2 sets (initial and follow-up) VR iP&ET done 5. PSA group: 19 rehabilitees only 1 set of VR iP&ET test

TABLE 1
Descriptive statistics of groups of participants
involved in VR iP&ET (N = 625)

| Group | Healthy Group | PSA Group |
|---|---|---|
| Group 1 (Secondary School students) | | Not Applicable |
| Group 2 (Tertiary school students) | 500 | |
| Group 3 (High-risk youth) | | |
| PSA group (Rehabilitee) | Not Applicable | 106 |
| (Completed two VR iP&ET test) | | |
| PSA group (Rehabilitee) | | 19 |
| (Completed one VR iP&ET test) | | |

For Group 1 (secondary school student), Group 2 (tertiary student), and Group 3 (high-risk youth), participants were required to provide basic demographic information, sign the consent (for Group 1 participants, the consent form will be signed by parents or guardians), and finish Pre-VR and Post-VR questionnaire and eye examination datasheet. For PSA group, rehabilitees had to fill in additional information, including drug use status, Chinese Drug Involvement Scale (C-DIS) form.

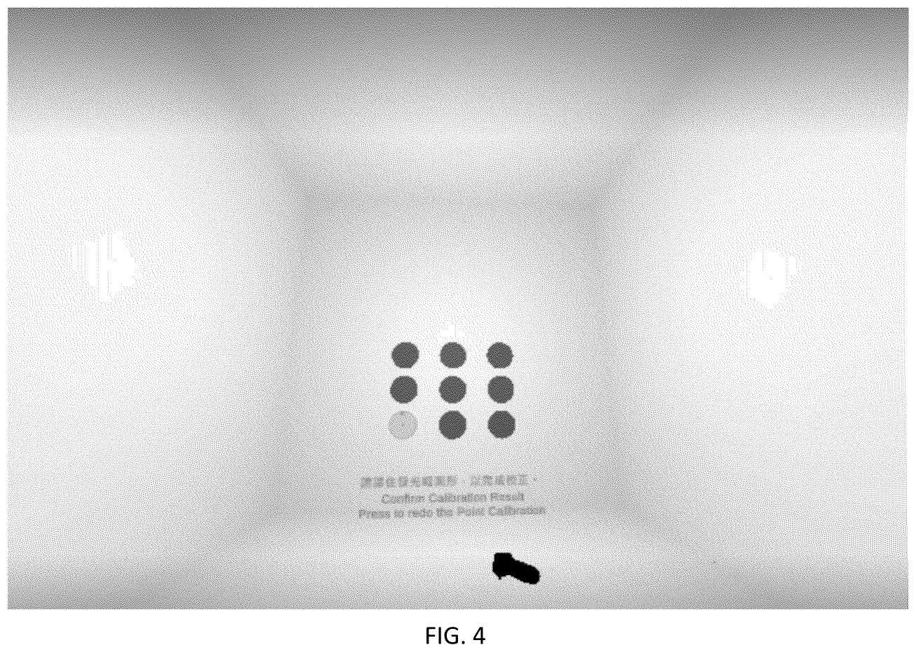

After finishing all documents, participants wore the VR headset and were asked to follow instructions to complete calibration. Participants needed to follow the light blue (the bottom left) circle with their eyes as shown in FIG. 4, allowing the eye-tracking sensor to calibrate with the eyeball spatial position and ensure the accuracy of data collected via eye-tracking sensor.

After calibration, the VR display shows 3 cycles of light stage, where the protocol of cycles of light stage is shown in Table 2. Subjects' pupil size will be captured with 120 Hz sampling rate via Infrared pupillometry in VR headset.

TABLE 2
Protocol of 3 cycles of light stage

| | | | Duration | |
|---|---|---|---|---|
| Stage | Stage: | PLR/RGB | (second) | |
| 1^{st} | Dark Stage | (PLR 0) (RGB 0, 0, 0) | 13 | s |
| 1^{st} | Low light | (PLR 64) (RGB 64, | 2 | s |
| | intensity | 64, 64) | | |
| 2^{nd} | Dark Stage | (PLR 0) (RGB 0, 0, 0) | 13 | s |
| 2^{nd} | Medium light | (PLR 128) (RGB 128, | 2 | s |
| | intensity | 128, 128) | | |
| 3^{rd} | Dark Stage | (PLR 0) (RGB 0, 0, 0) | 13 | s |
| 3^{rd} | High light | (PLR 255) (RGB 255, | 2 | s |
| | intensity | 255, 255) | | |
| 4^{th} | Dark Stage | (PLR 0) (RGB 0, 0, 0) | 13 | s |

After 3 stages of light stimuli, 13 sets of pictures will be shown, including 3 sets of control picture and 10 sets picture with either normal or drug related content. Under the eye-tracking part, the environment was kept quiet and comfortable to let subjects to undergo the test in a relaxed manner. And each subject is instructed to look freely around to minimize any bias caused by unintentional gaze on either type of picture.

In addition, a 5-minute VR educational video based on real-case referral from Department of Psychiatry, the Chinese University of Hong Kong, was provided after iP and ET test. The video content includes a real-case story, directed by an ex-drug addict to convey the message of mental and physical harm caused by drug abuse.

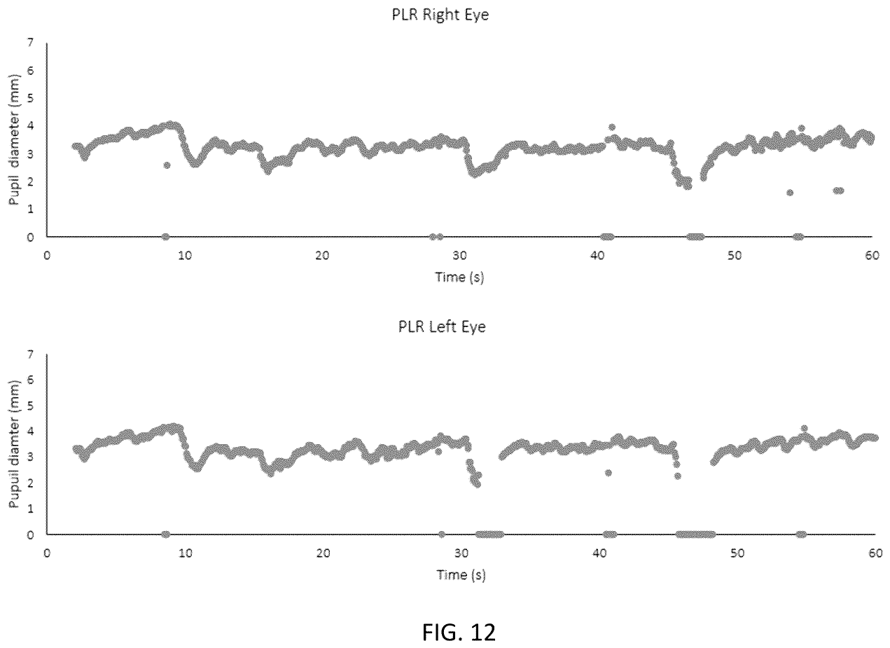

All pupillometry data evaluated by 3 masked observers is categorized into either normal pupillary response (NPR) or pupillary unrest (PU). The observers were instructed to grade the pupillometry graphs with the standard example of NPR and PU (standard example 1, 2, 3, 4 with FIG. 12-15) and reference to following criteria.

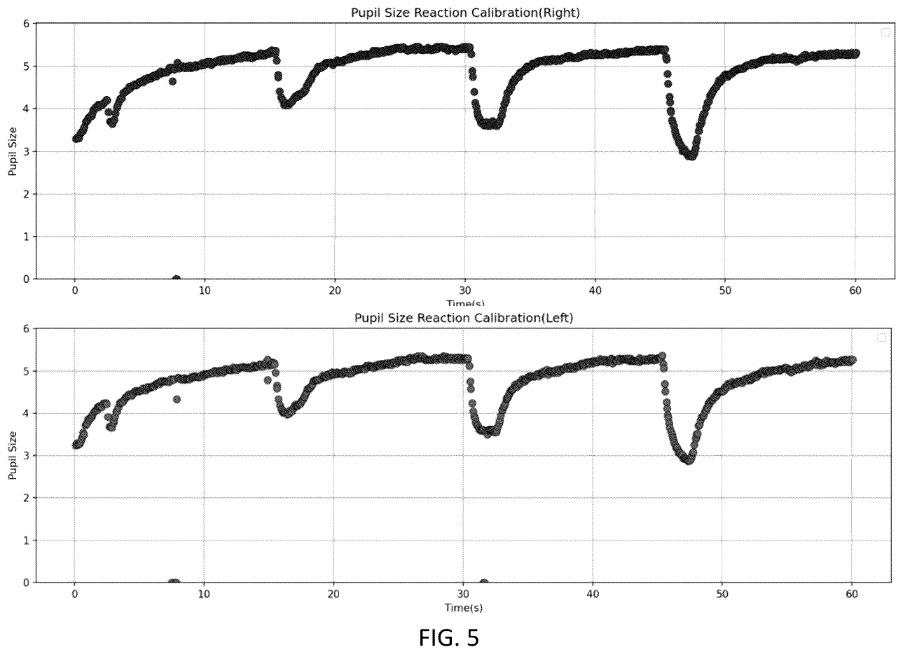

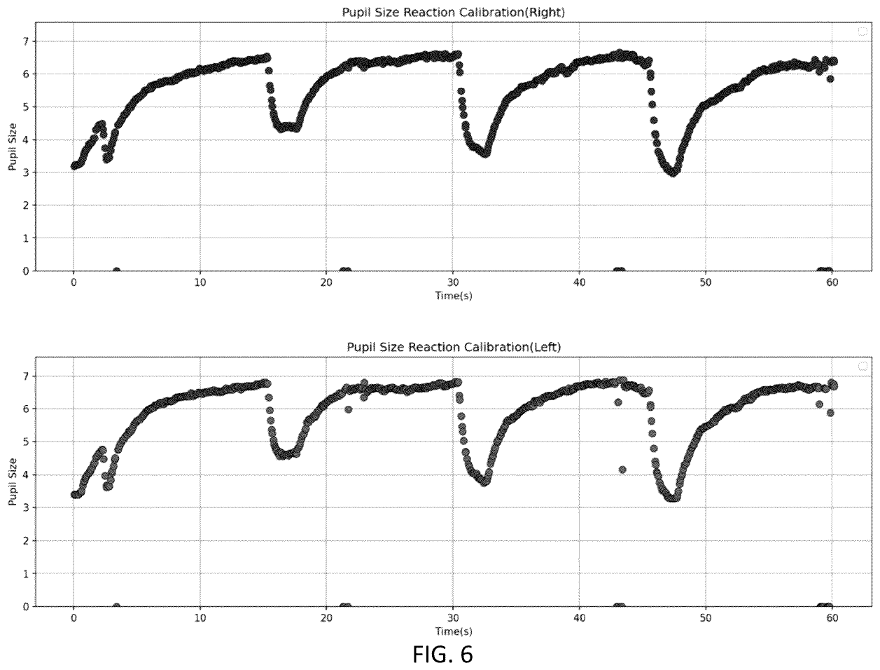

With reference to FIGS. 5 and 6, there are provided 2 examples of normal pupillary response (NPR) graph shows the following characteristics and criteria:
1. Steady pupil constriction and re-dilatation at the end of the light stage
2. Smooth pupil dilation during dark stage
3. Shorter time to recover to baseline of pupil size after each light stage

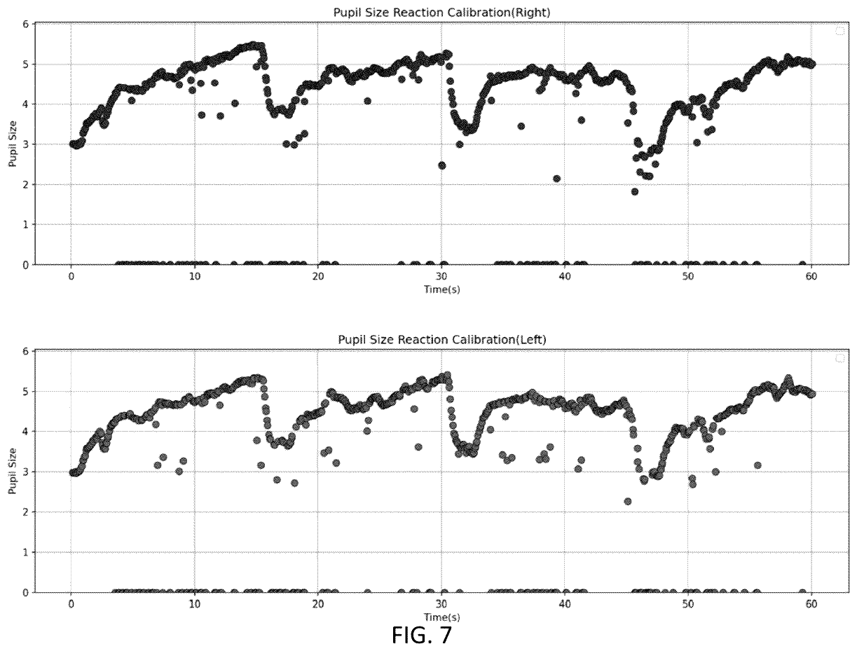

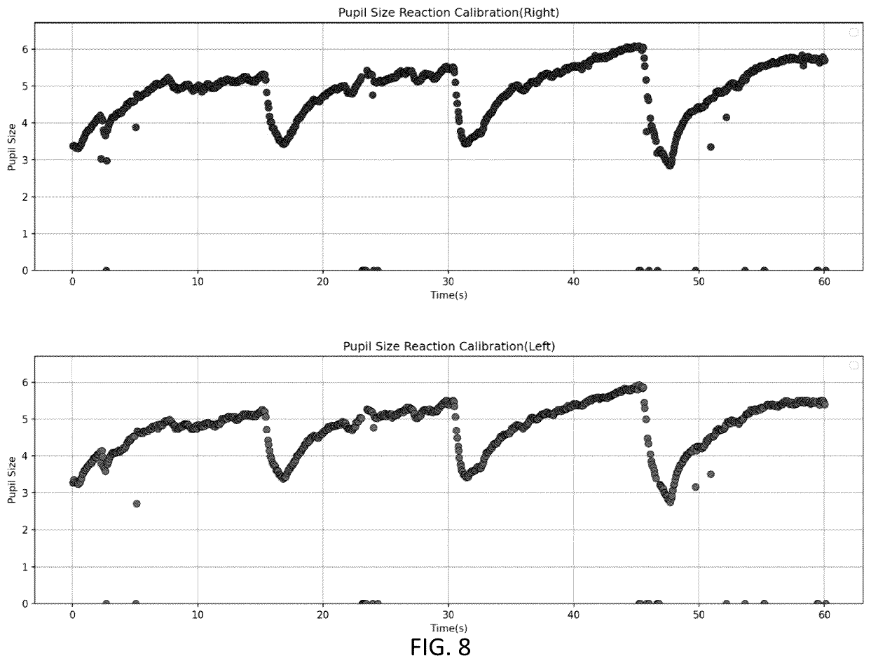

With reference to FIGS. 7 and 8, there are provided 2 examples of pupillary unrest (PU) in pupillometry tracing of both eyes from a poly-drug subject with 15 years history of PSA, which shows the following characteristics and criteria:
1. Fluctuating pupil constriction and dilatation
2. Unrest pupil dilatation during dark stage
3. Longer time to recover to baseline pupil size after light stage
4. Latency

Tables 3a and 3b further describe statistics of normal pupillary response and pupillary unrest in different groups.

TABLE 3a
Descriptive statistics of normal pupillary response
and pupillary unrest in different groups

| | | |
|---|---|---|
| | Normal pupillary | Pupillary |
| Group | response (N, %) | unrest (N, %) |

Healthy

| | | | | |
|---|---|---|---|---|
| Group 1 (N = 300) | 291 | (97%) | 9 | (2.6%) |
| Group 2 (N = 100) | 99 | (99%) | 1 | (1%) |
| Group 3 (N = 100) | 92 | (92%) | 8 | (8%) |
| Total (N = 500) | 482 | (96.4%) | 18 | (3.6%) |

Rehabilitee

| | | | | |
|---|---|---|---|---|
| 1^{st} visit (N = 125) | 6 | (4.8%) | 119 | (95.2%) |
| 2^{nd} visit (N = 106) | 7 | (6.6%) | 99 | (93.3%) |

TABLE 3b
Descriptive statistics of normal pupillary response
and pupillary unrest in different groups

| | Healthy | | Chi-Square |
|---|---|---|---|
| Group | Group | Rehabilitee | (P value) |
| Normal pupillary | 482 | 13 | 595.51 |
| response | | | (P < 0.05) |
| Pupillary | 18 | 218 | |
| unrest (PU) | | | |
| Total | 500 | 231 | |

All subject pupil size captured during VR iP&ET test with 120 Hz sampling rate. And there were three light stages of different intensity of light stimuli (PLR 64, 128, 255).

In Table 4b, the mean of Initial pupil size of healthy group in 1 stage (PLR 64) is 6.08 mm, σ=0.7, where PSA group is 4.40 mm, σ=0.86, P(T<=t) two-tail=0.017 (p<0.05).

Similar result shown in mean of maximum and minimum pupil size of heathy group in 1^{st} Stage (PLR 64) is 6.22 mm and 4.25 mm, σ=0.6 and σ=0.62, while in PSA group is 6.0 mm and 3.56 mm, σ=0.77 and σ=0.65, P(T<=t) two-tail=0.015 (p<0.05) and 0.4.

And in Table 4a, there is a notable difference (38.49%) of 1^{st} stage initial pupil size between healthy and PSA group, and the difference of initial pupil size in 2^{nd} and 3^{rd} stage initial pupil size is widening (42.14% and 69.06%).

And overall constriction power of healthy group is greater than PSA group by 3-5%, meaning overall healthy group subjects have bigger size of pupil size and better ability in pupil constriction. This can be explained by the type of drugs taken by the PSA, which will be further illustrated in the discussion section.

TABLE 4a
Descriptive statistics of pupil size between healthy and PSA groups

| | Healthy | PSA | |
|---|---|---|---|
| | Group | Group | |
| Parameter | (N = 500) | (N = 106) | Difference |

1^{st} Stage (PLR 64)

| | | | | |
|---|---|---|---|---|
| Initial pupil size (mm) | 6.08 | 4.40 | 1.69 | (38.49%) |
| Maximum pupil size (mm) | 6.22 | 6.00 | 0.21 | (3.5%) |
| Minimum pupil size (mm) | 4.25 | 3.56 | 0.69 | (19.43%) |

| | | | |
|---|---|---|---|
| Constriction power | 31.70% | 32.40% | 0.07% |

2^{nd} Stage (PLR 128)

| | | | | |
|---|---|---|---|---|
| Initial pupil size (mm) | 6.24 | 4.40 | 1.85 | (42.14%) |
| Maximum pupil size (mm) | 6.38 | 6.13 | 0.24 | (3.9%) |
| Minimum pupil size (mm) | 3.62 | 3.37 | 0.25 | (7.41%) |

| | | | |
|---|---|---|---|
| Constriction power | 43.14% | 39.48% | 3.66% |

3^{rd} Stage (PLR 255)

| | | | | |
|---|---|---|---|---|
| Initial pupil size (mm) | 6.12 | 3.63 | 2.5 | (69.06%) |
| Maximum pupil size (mm) | 6.32 | 6.02 | 0.31 | (5.14%) |
| Minimum pupil size (mm) | 3.11 | 2.94 | 0.16 | (5.44%) |

| | | | |
|---|---|---|---|
| Constriction power | 50.85% | 45.88% | 4.97% |

TABLE 4b
Descriptive statistics of subjects characteristics
with pupil size between healthy and PSA groups

| | | | p-value (Two |
|---|---|---|---|
| | Heathy Group | PSA Group | Tailed Test) |
| Subjects (n) | 500 | 106 | |

Gender

| | | | |
|---|---|---|---|
| Male | 237 (47.4%) | 88 (83.0%) | 0.12 |
| Female | 263 (52.6%) | 18 (17.0%) | |
| Mean Age, years ± SD | 19.46 ± 8.74 | 47.95 ± 18.50 | |
| Positive Urine test | 0 (0%) | 29 (27.36%) | |
| result (%) | | | |

Initial pupil size (mm)
Mean ± SD (95% CI)

| | | | |
|---|---|---|---|
| 1^{st} Stage (PLR 64) | 6.08 ± 0.70 (5.94, 6.23) | 4.40 ± 0.86 (4.25, 4.55) | |
| 2^{nd} Stage (PLR 128) | 6.24 ± 0.68 (6.10, 6.39) | 4.40 ± 0.89 (4.24, 4.56) | 0.017 |
| 3^{rd} Stage (PLR 255) | 6.12 ± 0.79 (5.95, 6.29) | 3.63 ± 1.12 (3.43, 3.82) | |

Maximum pupil size (mm)
Mean ± SD (95% CI)

| | | | |
|---|---|---|---|
| 1^{st} Stage (PLR 64) | 6.22 ± 0.60 (6.09, 6.35) | 6.00 ± 0.77 (5.87, 6.14) | |
| 2^{nd} Stage (PLR 128) | 6.38 ± 0.63 (6.24, 6.51) | 6.13 ± 0.8 (5.99, 6.27) | 0.014 |
| 3^{rd} Stage (PLR 255) | 6.32 ± 0.62 (6.20, 6.46) | 6.02 ± 0.97 (5.85, 6.19) | |

Minimum pupil size (mm)
Mean ± SD (95% CI)

| | | | |
|---|---|---|---|
| 1^{st} Stage (PLR 64) | 4.25 ± 0.62 (4.11, 4.38) | 3.56 ± 0.65 (3.45, 3.67) | |
| 2^{nd} Stage (PLR 128) | 3.62 ± 0.54 (3.51, 3.74) | 3.38 ± 0.60 (3.27, 4.48) | 0.40 |
| 3^{rd} Stage (PLR 255) | 3.11 ± 0.47 (3.00, 3.21) | 2.94 ± 0.62 (2.83, 3.05) | |

Eye Tracking (ET) of the subject is performed on 13 sets of pictures including 3 sets of control and 10 sets of pictures with either normal or drug-related content. the subject's first sight, duration and total number of attentions towards two types of pictures were recorded.

At first, the subject will look at a book which displays 3 sets of non-drug related pictures on both the left- and right-hand side. Then there are 10 sets of pictures, with one being drug-related picture and one being similar picture but without drug-related content. The drug-related picture will be shown on either the left or right side of the book. The system will record the subject's first attention on either drug-related picture or normal control picture, and the average number of first sight on 10 sets of drug-related picture or normal picture as following (First sight) was calculated. Also, VR iP&ET system will record the total duration of time (second) and total number of attentions on normal picture or drug-related picture. The higher score at first sight, durations and total number of attentions suggests the implication of higher tendency or higher risks on substance abuse (Saladin et al., 2006).

In table 6, it is similar result of first sight with normal or drug-related picture in healthy and PSA group (3.9%, 4%). But it is obvious to note the significant difference of gaze duration on normal or drug-related pictures in healthy and PSA group, suggesting PSA group have longer duration of gaze on drug-related picture compared to healthy group.

TABLE 5
First sight, duration, and total number of attentions in different groups of subjects

| | | | | | | | Total number of | | |
|---|---|---|---|---|---|---|---|---|---|
| | First Sight | | | Duration (s) | | | attentions | | |
| | Normal | Drug | Change | Normal | Drug | Change | Normal | Drug | Change |
| Healthy | | | | | | | | | |
| Group | | | | | | | | | |
| Group 1 | 0.52 | 0.45 | 0.07 | 2.21 | 1.92 | 0.29 | 50.46 | 44.09 | 6.37 |
| | | | (15%) | | | (15%) | | | (14.4%) |
| Group 2 | 0.48 | 0.51 | 0.03 | 2.32 | 2.02 | 0.3 | 51.80 | 45.28 | 6.52 |
| | | | (5.8%) | | | (14.8%) | | | (14.4%) |
| Group 3 | 0.52 | 0.50 | 0.02 | 3.51 | 1.98 | 1.53 | 52.85 | 48.82 | 4.03 |
| | | | (4%) | | | (77%) | | | (8.3%) |
| Average | 0.51 | 0.48 | 0.03 | 2.68 | 1.97 | 0.71 | 51.70 | 46.06 | 5.64 |
| | | | (6.3%) | | | (36%) | | | (12.2%) |
| PSA | | | | | | | | | |
| Group | | | | | | | | | |
| 1^{st} Visit | 0.49 | 0.50 | 0.01 | 2.13 | 2.16 | 0.03 | 48.92 | 49.98 | 1.06 |
| | | | (2%) | | | (1.3%) | | | (2.1%) |
| 2^{nd} Visit | 0.49 | 0.50 | 0.01 | 2.00 | 2.21 | 0.21 | 47.86 | 52.37 | 4.51 |
| | | | (2%) | | | (9.5%) | | | (8.6%) |
| Average | 0.49 | 0.50 | 0.01 | 2.06 | 2.18 | 0.12 | 48.39 | 51.17 | 2.78 |
| | | | (2%) | | | (5.5%) | | | (5.4%) |

TABLE 6
Comparison of first sight, duration, and total number of
attentions between the healthy group and the PSA group

| | | |
|---|---|---|
| Healthy | PSA | Difference |

First sight

| | | | | |
|---|---|---|---|---|
| Normal | 0.51 | 0.49 | 0.02 | (3.9%) |
| Drug | 0.48 | 0.50 | 0.02 | (4%) |
| Duration (s) | 2.68 | 2.06 | 0.62 | (23%) |
| Normal | | | | |
| Drug | 1.97 | 2.18 | 0.21 | (11%) |

Total number of attentions

| | | | | |
|---|---|---|---|---|
| Normal | 51.70 | 48.39 | 3.31 | (6.4%) |
| Drug | 46.06 | 51.17 | 0.21 | (10.6%) |

In the experiment, 606 sets of pupillometry data were collected. In preliminary analysis, the pupil size along the time with several light stimuli was analysed. Three independent assessors were instructed to classify the graphs into normal pupillary response and pupillary unrest with reference to the standard example (FIG. 12-15). It was found that 96.4% (N=482) of subjects fall into the NPR category and 3.6% (N=18) of their graphs have PU in healthy group.

While in PSA group, there were 95.2% (N=119) and 93.5% (N=99) subjects having PU in 1^{st} visit and 2^{nd} visit, respectively.

Normal pupil size in adults varies from 2 to 4 mm in bright light and 4 to 8 mm in dark environment. The initial pupil size (mm), maximum pupil size (mm), minimum pupil size (mm) were recorded during the VR iP&ET test under the three light stages. The interpretation of pupil size and its implication is in Table 7.

With the formula and calculation, the constriction power of healthy group and PSA group in different light stages, where constriction power implicated the maximum degree of pupil constriction in a fixed duration.

TABLE 7
Pupil size and its implication

| | | Environment | | Normal |
|---|---|---|---|---|
| Pupil Size | Phase | setting | Implication | Range |
| Initial | Dilatation/ | First second of | The extent of | 4-8 mm |
| pupil size | Recovery | light stimuli | recovery after | |
| | Phase | in each stage | light stimuli | |
| Maximum | Dilatation/ | 13 second of | The greatest | 4-8 mm |
| pupil size | Recovery | darkness | extent of pupil | |
| | Phase | (PLR 0) | dilatation under | |
| | | | dark environment | |
| Minimum | Constriction | During 2 | The greatest | 2-4 mm |
| | | seconds of | extent of pupil | |
| pupil size | phase | light stage | constriction under | |
| | | (PLR 64, 128, | light stimuli | |
| | | 255) | | |

### Example 2

### Participant

This study was conducted at the Chinese University of Hong Kong Eye Centre (CUHKEC), Hong Kong, China from January to October 2021. Total 233 participants (188 men and 45 women), with ages ranging from 15 to 79 were recruited via the Chinese University of Hong Kong (CUHK) intranet mass mail and local counseling centres for psychoactive substance abusers. The target participants were categorized as two groups, including psychoactive substance abuser group (PSA, n=97) and healthy control group (HC, n=136) (Table 8). All individuals were invited to the centre for a 1-day visit to undergo ophthalmic investigations. All procedures and measures of this study were performed in compliance with the Joint CUHK-New Territories East Cluster Clinical Research Ethics Committee (Ref No.: 2019.007). Prior to testing, written informed consent was obtained from all participants in the study. All data was kept strictly confidential for research purposes.

TABLE 8
Demographics of the health controls (HC) and the psychoactive
substance abusers (PSA)

| | HC (n = 136) | | | PSA (n = 97) | | |
|---|---|---|---|---|---|---|
| Variables | n | % | M ± SD | n | % | M ± SD |
| Sex | | | | | | |
| Male | 106 | 77.94 | | 82 | 84.53 | |
| Age | | | 45.05 ± 17.00 | | | 46.74 ± 19.04 |
| Sex | | | | | | |
| Female | 30 | 22.05 | | 15 | 15.46 | |
| Age | | | 39.10 ± 17.79 | | | 38.14 ± 17.94 |

Inclusion criteria included, 1) normal visual acuity or corrected vision, and 2) without any known ophthalmic disorders (e.g. uveitis, limitation in extraocular movement), neurological disorders (e.g. cerebrovascular disease) and systemic disorders (e.g. diabetes mellitus). Subjects with previous ocular or traumatic brain injuries were not recruited in this study. In addition, participants of the PSA group were selected with a self-reported psychoactive drug use history and frequency in recent 6 months, including cannabis, cocaine, heroin, ketamine and methamphetamine (at least one of these five drugs). There were no age and sex limits.

### Demographics and Substance Use

Demographic information was collected by a self-report questionnaire prior to the study. An additional questionnaire was given to the participants in the PSA group for collecting the history of substance use, frequency and involvement. Instead of using conventional pupillometric devices, this study implemented the use of VR HMD integrated with an eye-tracking add-on. IP data was measured by the equipment comprising HTC Cosmos HMD, with field of view and six degrees of freedom, 1440×1700 pixels per eye resolution and sampling rate of 90 Hz. Binocular Droolon F1 Eye-tracking add-on was installed with less than 0.5-degree accuracy, less than 5 ms delay and sampling rate of 120 Hz. VR experiment processes were designed with reference to HTC VIVE VR safety and regulatory guidelines.

### Urine Toxicology

The collections of urine, hair and saliva are the most commonly used methods for substance detection, for verification of results obtained by the system. Each has a particular advantage and disadvantage. As a flexible and inexpensive method, urine toxicology could detect a wide range of drugs. Definitive test and point of care test are the two basic types. For the definitive test, there are two processes performed in the laboratory. The initial screening is processed by immunoassay technology, and presumed positive results would be further confirmed by gas chromatography/mass spectrometry (GC/MS) or liquid chromatography/mass spectrometry (LC/MS). Urinary excretion rates and duration may lead to different windows of detection for drugs. Moreover, the detection time is subject to the frequency of drug use. The detection windows for casual and chronic users are 4 days and more than 7 days respectively. With regards to common psychoactive substances in this locality, the urine toxicology can detect opioids, cocaine and methamphetamine in the urine for approximately 3 days after use.

Each participant's urine sample was collected and sent for a comprehensive drug screening per visit. Over 100 types of drugs and their metabolites present in urine were analyzed by liquid chromatography time-of-flight mass spectrometry (LC/MS) in the Department of Chemical Pathology at the Chinese University of Hong Kong.

### Statistical Analysis

All statistical analyses were performed using SPSS (Statistical Package for Social Sciences Inc., Chicago, Illinois, USA) version 27.0. Independent sample t-test was used to compare means from two unpaired groups. One-way analysis of variance (ANOVA) was performed for comparison amongst three or more unpaired groups. Statistical difference was considered significant if P<0.05 and 95% confidence interval (95% CI) was calculated. The variational modes in the functional data between PSA and HC were facilitated by functional principal component analysis (PCA). Receiver operating characteristic (ROC) analysis was conducted for the comparison of the area under the curve (AUC) for PSA and HC groups. Continuous registration, smoothing spline, PCA and ROC were facilitated by packages in R version 4.1.1. Data points for each sample were calculated by taking the average of the data of the two eyes. Since sequences have various lengths, the linear interpolation was utilized to guarantee equal length in every sequence. PLR was visualized using this study by infrared pupillometry.

### Study Population

233 participants (188 men and 45 women), with age ranging from 15 to 79 (44.49±17.977) were categorized into two groups, including psychoactive substance abuser group (n=97; aged between 15 and 79, 45.55±18.943) and healthy control group (n=136; aged between 16 and 73, 43.74±17.29). For the PSA group, cannabis (n=40; aged between 15 and 66, 30.55±14.35), cocaine (n=12; aged between 16 and 57, 30.83±13.49), heroin (n=50; aged between 40 and 79, 60.92±10.03), ketamine (n=18; aged between 16 and 67, 40.11±17.37), methamphetamine (n=24; aged between 22 and 70, 43.88±11.16) and others (n=18; aged between 16 and 41.83±16.87) were further categorized. 38.14% of the recruited subjects (n=37; aged between 16 and 70, 41.49±16.16) in the PSA group had more than one psychoactive substance abuse, and therefore were referred as poly-drug users (see table 9). The average year of substance use is 19 years in the PSA group.

TABLE 9
Types and duration of drug use amongst the
psychoactive substance abusers (PSA)

| | | |
|---|---|---|
| Variables | n | % |

Types of drugs taken

| | | |
|---|---|---|
| Cannabis | 40 | 41.24 |
| Ketamine | 18 | 18.56 |
| Cocaine | 12 | 12.37 |
| Methamphetamine | 24 | 24.74 |
| Heroin | 50 | 51.55 |
| Others | 18 | 18.56 |
| Poly drug user | 37 | 38.14 |

Duration of taking drug (years)

| | | | |
|---|---|---|---|
| ≤1 | year | 13 | 13.40 |
| 2-15 | years | 34 | 35.05 |
| 16-29 | years | 24 | 24.74 |
| ≥30 | years | 26 | 26.80 |

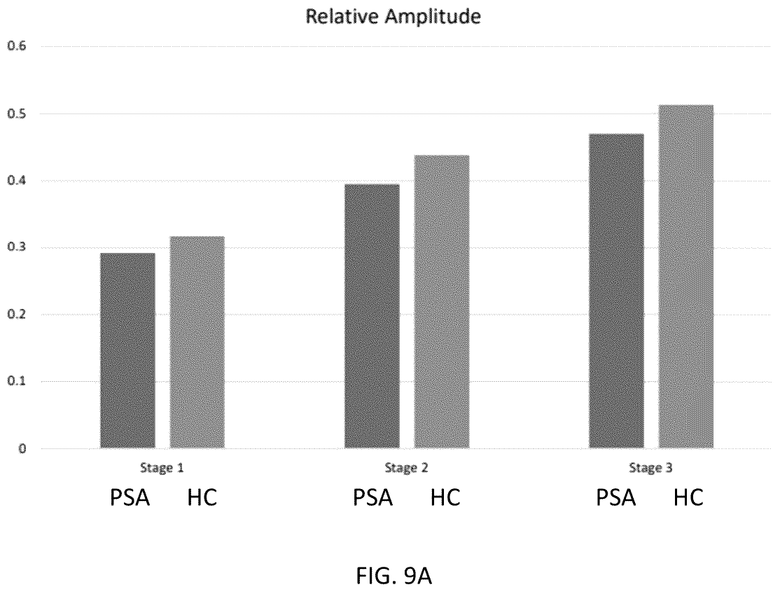

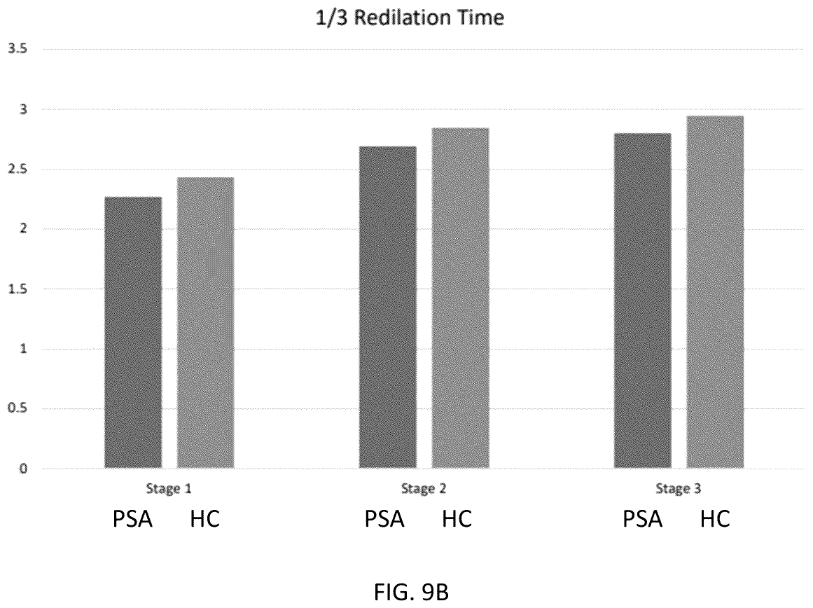

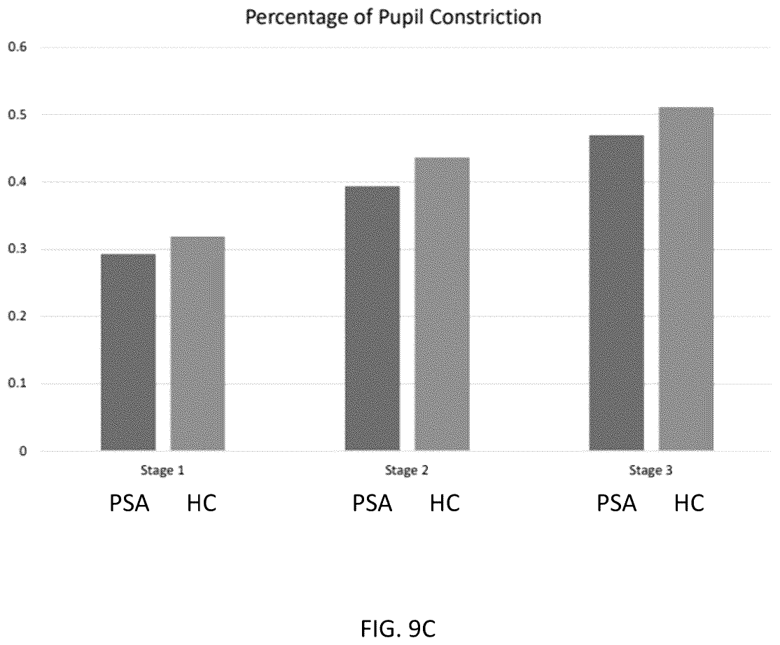

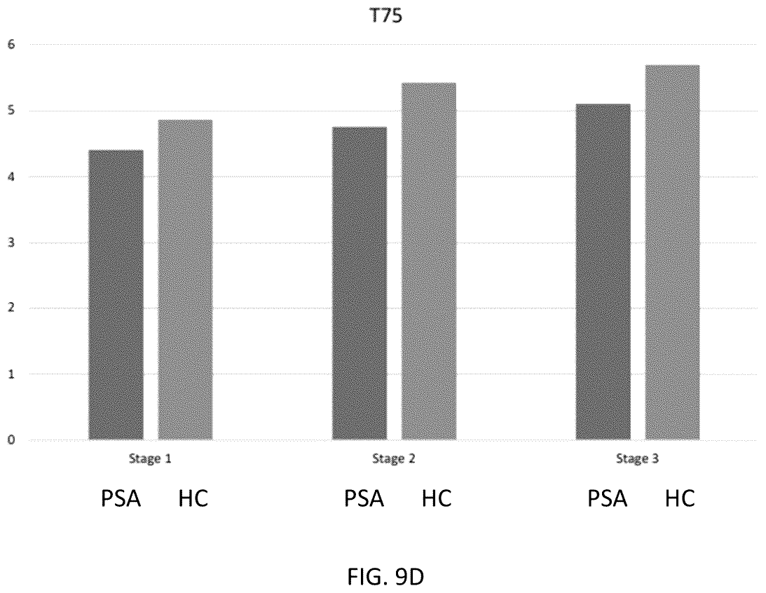

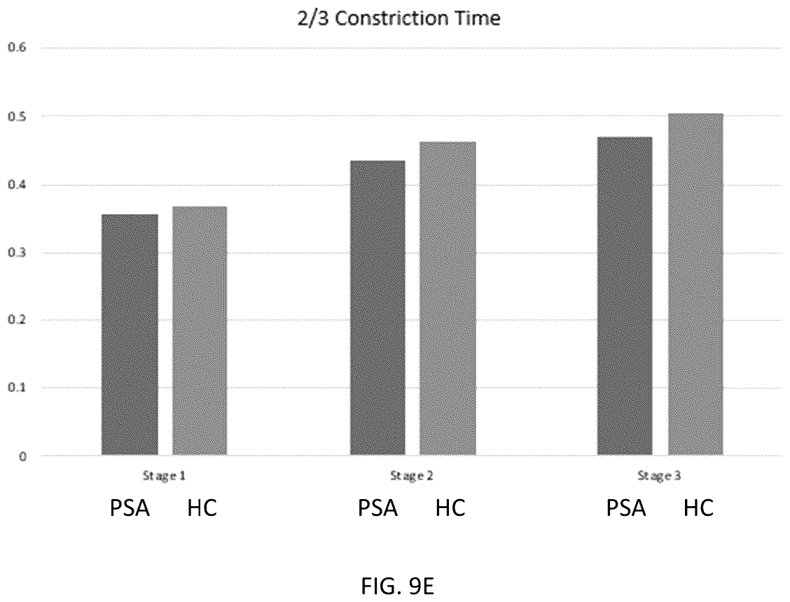

With reference to FIGS. 9A to 9E, there is shown comparison of the relative amplitude, ⅓ redilation time, percentage of pupil constriction, recovery time to achieve 75% of maximal pupil size (T75) and % constriction time between the psychoactive substance abusers (blue) and the health controls (orange).

### Comparisons Between Psychoactive Substance Abusers (PSA) and Health Control (HC)

There were significant statistical difference in all PLR light stages between the healthy control group and the psychoactive substance abuser group in 4 key parameters, namely relative amplitude, ⅓ redilation time, percentage of pupil constriction and recovery time to achieve 75% of maximal pupil size (T75).

The relative amplitude of the PSA group was significantly lower in all 3 PLR stages compared to the HC group. In PLR 64, the mean (SD) relative amplitude of the PSA group and HC group were 0.291% (0.063) and 0.317% (0.051) respectively. There was a significant difference at PLR 64 (p<0.001). In PLR 128, that of the PSA group and HC group were 0.395% (0.076) and 0.438% (0.048). This parameter was significantly lower in the PSA group (p<0.001). In PLR 255, the PSA group demonstrated 0.467% (0.059) in relative amplitude whereas the HC group showed 0.513% (0.040). The PSA group once again showed significantly lower relative amplitude (p<0.001).

The ⅔ constriction time was also noted to be significantly quicker in the PSA group at PLR 64 (p=0.026), PLR 128 (p=0.005) and PLR 255 (p<0.001). For the first light stage, the mean (SD) ⅔ constriction time was 0.356 (0.039) seconds and 0.369 (0.044) seconds for PSA and HC accordingly. In the second stage, it was 0.436 (0.080) seconds and 0.462 (0.064) seconds. In the final light stage, it was 0.470 (0.080) seconds and 0.505 (0.070) seconds respectively.

Moreover, there was a significantly smaller percentage of pupil constriction in the PSA group in all light stages. In PLR 64, the mean (SD) was 0.292 (0.062)% and 0.319 (0.042)% in the PSA group and the HC group respectively (p<0.001). In PLR 128, that of the PSA group and the HC group were 0.394 (0.075)% and 0.436 (0.047)% separately (p<0.001). In PLR 255, the PSA group was noted with 0.470 (0.058)% which was significantly lower than the HC group's 0.511 (0.039)% (p<0.001).

The ⅓ redilation time was another crucial parameter in this study as it consistently demonstrated a lower value in the PSA group compared to the HC group regardless of the light intensity. In PLR 64, the mean ⅓ redilation time (SD) of the PSA group and the HC group were 2.267 (0.512) seconds and 2.430 (0.435) seconds correspondingly. The PSA group displayed a significantly lower level (p=0.012) than the HC group. In PLR 128, that of the PSA group and the HC group were 2.690 (0.209) seconds and 2.845 (0.173) seconds respectively. The HC group obtained a significantly higher value (p<0.001). In PLR 255, the PSA group achieved a significantly quicker redilation time at 2.799 (0.212) seconds compared to 2.943 (0.119) seconds in the HC group (p<0.001).

The recovery time to achieve 75% of maximal pupil size (T75) was the fifth parameter that could significantly differentiate the PSA group from the HC group with a shorter time in all 3 light stages. In PLR 64, the PSA group had a significantly shorter 4.405 (SD: 1.018) seconds while that of HC group was 4.861 (0.847) seconds (p<0.001). In PLR 128, T75 in the PSA group and the HC group were 4.753 (0.806) seconds and 5.423 (0.841) seconds respectively. The PSA group achieved a significantly shorter T75 recovery time than the HC group (p<0.001). Lastly, the T75 in the PSA group was 5.101 (1.009) seconds which was significantly quicker than 5.692 (0.839) seconds obtained by the HC group (p<0.001).

In addition, the response time was shown significantly faster in the PSA group when compared to the HC group in PLR 128 (p=0.005) and PLR 255 (p<0.001). However, pupil latency, average constriction velocity, dilation velocity in the rapid and slow phases were not significant parameters to differentiate the PSA group from the HC group.

### Comparisons Between Heroin Users (HU) and Health Control (HC)

There were 4 parameters with consistent statistical difference between the health control group and the heroin user group in all light stages. Relative amplitude of the HU group was found to be significantly lower in 3 PLR stages (all p<0.001) when compared to the HC group. The mean (SD) of the HU and the HC group was 0.268 (0.050)% and 0.317 (0.051)% in the first light stage. For the second light stage, it was 0.363 (0.052)% and 0.438 (0.048)% accordingly. For the brightest light stage, the HU group still delivered a lower 0.441 (0.049)% amplitude than the HC group which was 0.513 (0.040)%.

Percentage of pupil constriction was another parameter to differentiate between the HU group from the HC group (all p<0.001). In PLR 64, the HU group was noted with a significantly lower mean percentage (SD) of 0.269 (0.051)% than 0.319 (0.049)% in the HC group. In PLR 128, it was 0.363 (0.054)% and 0.436 (0.047)% for the HU and HC groups respectively. In PLR 255, it was 0.441 (0.050)% and 0.511 (0.039)%.

The HU group also achieved a significantly quicker ⅓ redilation time than the HC group in all three light stages. For the first light stage, the mean (SD) was 2.219 (0.488) seconds for the HU group and 2.430 (0.435) seconds for the HC group (p=0.007). In the second light stage, it was 2.638 (0.225) seconds and 2.845 (0.173) seconds (p<0.001) for the HU and HC group respectively. In the final light stage, the ⅓ redilation time was 2.780 (0.192) seconds for the HU group. This was shorter than 2.943 (0.119) seconds in the HC group (p<0.001).

T75 was identified to be faster in the HU group than the HC group in all light stages (all p<0.001). The mean (SD) was 4.318 (0.955) seconds for the HU group and 4.861 (0.847) seconds for the HC group in the first light stage. In the second light stage, it was 4.644 (0.773) seconds and 5.423 (0.871) seconds in the two groups respectively. In the last light stage, the mean (SD) was 4.918 (0.914) seconds for the HU group and 5.692 (0.839) seconds for the HC group.

### Comparisons Between Cannabis Users (CU) and Health Control (HC)

The ⅓ redilation time for subjects with cannabis use was significantly shorter than the HC group in the second and third light stages. For PLR 128, the mean ⅓ redilation time (SD) was 2.745 (0.209) seconds in the CU group which was less than 2.845 (0.173) seconds in the HC group. For PLR 255, it was 2.814 (0.271) seconds and 2.943 (0.119) seconds for the CU and HC groups respectively. The mean was consistently lower in PLR 64 although there was no statistical significance.

### Comparisons Between Cannabis Users (CU) and Heroin Users (HU)

The relative amplitude, percentage of pupil constriction, T75 recovery time and dilation velocity in the rapid phase were the 4 parameters to demonstrate statistical difference between the HU and the CU groups.

The relative amplitude was shown to be consistently greater in the CU group compared to the HU group regardless of the light intensity. In PLR 64, the mean (SD) relative amplitude was 0.318 (0.063)% and 0.268 (0.050)% in the CU and HU groups respectively. In PLR 128, that of the CU and HU groups were 0.436 (0.066)% and 0.363 (0.052)%. In PLR 255, the CU group achieved 0.450 (0.052)% while the HU group obtained 0.441 (0.049)%. In all 3 light stages, the relative amplitude in the CU group was larger than that of the HU group (all p<0.001).

The dilation velocity in the rapid phase was a new parameter noted with statistical significance when the comparison was made between the CU and HU groups but not between the PSA and HC groups. In PLR 64, the velocity in the CU and HU groups was 0.780 (0.450) and 0.576 (0.317) millimeters per second individually. The CU group had a significantly faster speed (p=0.013). In PLR 128, that of the CU and HU groups was 1.344 (0.276) and 1.020 (0.533) millimeters per second, in which a significantly faster velocity was noted in the CU group (p<0.001). In the PLR 255, the CU group had a faster 1.396 (0.310) millimeters per second in speed as compared to 1.214 (0.797) millimeters per second in the HU group despite the lack of significance (p=0.065).

The percentage of pupil constriction was another consistent parameter where the HU group had a significantly smaller value than the CU group in all 3 light stages. In PLR 64, the mean (SD) percentage of the CU group was 0.319 (0.059)%. This was significantly greater than that of the HU group which was 0.269 (0.051)% (p<0.001). In PLR 128, the CU group achieved a significantly larger percentage of pupil constriction by 0.432 (0.069)% compared to 0.363 (0.054)% in the HU group (p<0.001). In PLR 255, that of the CU and HU groups were 0.498 (0.049)% and 0.441 (0.050)% correspondingly (p<0.001).

The T75 recovery time was also noted to differentiate the CU and HU groups. The recovery time was quicker in the CU group compared to the HU group in all three light stages. In PLR 255, the mean (SD) T75 recovery time was 5.356 (1.087) seconds and 4.918 (0.914) seconds in the CU and HU groups respectively (p=0.042). The p values in PLR 64 and 128 were however not significant.

### Comparisons Between Psychoactive Substance Abusers (PSA) and Health Control (HC) by Sex

Five different pupillometric parameters were found to be statistically different for male in the PSA and HC groups respectively. Male subjects in the PSA group demonstrated lower relative amplitude (PLR 64: p=0.006; PLR 128: p<0.001; PLR 255: p<0.001), quicker response time (PLR 64: p=0.015; PLR 128: p=0.002; PLR 255: p<0.001), shorter % constriction time (PLR 64: p=0.015; PLR 128: p=0.002; PLR 255: p<0.001), smaller percentage of pupil constriction (PLR 64: p=0.003; PLR 128: p<0.001; PLR 255: p<0.001) and quicker T75 (PLR 64, 128, 255: p<0.001). As for female, percentage of pupil constriction was found to be the only significant parameter that was smaller in the PSA groups in PLR 128 (p=0.022) and PLR 255 (p<0.001).

### Comparisons Between Psychoactive Substance Abusers (PSA) and Health Control (HC) by Age

All subjects in the PSA and HC groups were divided into 4 age groups (age 20, 21-40, 41-60, ≥61). Relative amplitude was shown to be significantly smaller in 2 groups of psychoactive substance abuser with elder age in all light stages (age 41-60, 61). For subjects aged 41-60, a smaller amplitude was noted in the PSA group (PLR 64: p=0.006; PLR 128: p<0.001; PLR 255: p<0.001). For subjects aged ≥61, similar comparison was achieved (PLR 64: p=0.002; PLR 128: p<0.001; PLR 255: p<0.001). Percentage of pupil constriction was another crucial parameter which was significantly smaller in the two elder PSA groups (age 41-60, 61).

### Pupillary Unrest

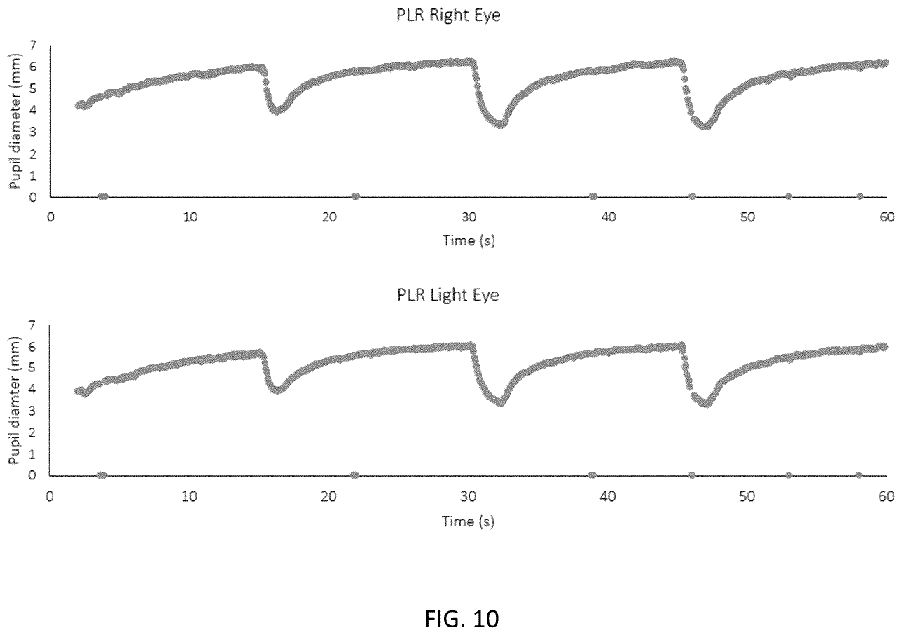

A typical pupillary light reflex (PLR) in the light stage registered by the infrared pupillometry in a normal subject was an acute slope curving downwards which signified pupillary constriction and hence reduction in pupil size (FIG. 10). In the dark stage, the pupil dilated back to its baseline diameter. A smooth curve was captured with a steep slope at the first ⅓ portion of the redilation phase, followed by a gradual flattening at the mid ⅓ portion, and plateauing smoothly at the last ⅓ portion. The majority of the HC group (88.971%; n=121) demonstrated this consistent phenomenon in every dark stage of the dark-light alternations in bilateral eyes.

Note. This was an infrared pupillography of a 24-year-old male with no history of substance abuse. The pupil dilated smoothly (curving upwards) from the first dark stage, and abruptly constricted in the first light stage. Similar patterns were seen in the second and third light stages in both eyes. (Orange: Right Eye; Blue: Left Eye)

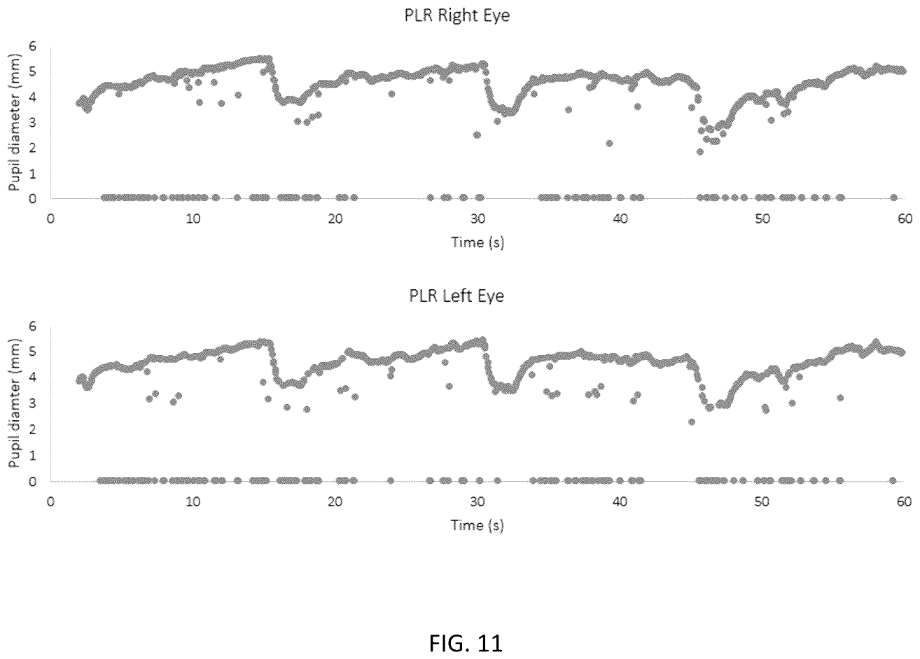

However, a remarkable 83.505% of the PSA group (n=81) were noted with a particular pattern of oscillating curve during the redilation phase of the dark stages. Pupillary unrest, also described as pupillary hippus, was captured in FIG. 11. The diameter of the pupil fluctuated during the dark stages, resulting in an unsmooth curve of redilation. The pupillary unrest was consistently captured in all three light stages (PLR 64, 128, 255). A normal pupillometry was however seen in 16.495% (n=16) of the PSA group where a smooth redilation curve was demonstrated and indistinguishable from that of normal subjects. The pattern of pupillary unrest was also noted in 11.029% (n=15) in the HC group without history of substance abuse, ophthalmic, neurological, or systemic disorder, rendering the possibility of physiological variation.

This was an infrared pupillography of a 41-year-old male with active use of heroin and history of multiple drug abuse. The pupil dilated in an oscillating manner with varying frequency and amplitude of waveform in the first dark stage, and then constricted in the first light stage. Similar patterns were seen in the second and third light stage in both eyes. (Orange: Right Eye; Blue: Left Eye) With reference to FIG. 12, while pupillary unrest was commonly seen among subjects with active or recent history of psychoactive substance abuse, there was an exception where a former psychoactive substance abuser who had withdrawn from PSA for at least 20 years exhibited a pupillary unrest similar to those of active substance abusers. There was no documented neurological, ophthalmic or systemic disease in this otherwise healthy individual. This example may suggest future studies on the immediate and long-term effect of psychoactive substance abuse in pupillary response.

### Urine Toxicology

A total of 97 urine samples from individuals identified as PSA were analyzed using liquid chromatography time-of-flight mass spectrometry (LC/MS). Out of these samples, 27 (27.8%) tested positive for the presence of drugs, while 70 (72.2%) tested negative.

The positive results indicated recent drug use in the individuals, providing evidence of short-term drug exposure. However, it is important to acknowledge the limitations of urine toxicology testing in providing long-term drug use results.

The self-reported drug use history provided by the participants indicated that they had used drugs at least once within the past 6 months. The urinary excretion rates and duration of drugs could vary, leading to different windows of detection for different substances. Furthermore, the urine toxicology test employed in this study was specifically focused on detecting opioids, cocaine, and methamphetamine, which are common psychoactive substances. The test had a detection window of approximately 3 days after use for these substances.

Therefore, while the LC/MS urine toxicology test displayed effectiveness in detecting recent drug use, it is important to consider the limitations associated with the detection windows for different drugs. Long-term drug use may not be accurately captured through urine testing alone.

### Area Under the Receiver Operating Characteristic Curve (AUC)

For the differentiating power of ROC in PLR 128 between PSA and ROC, the mean AUC of ⅓ redilation time was 0.736 which correlated with statistical significance (p<0.001) by independent sample t-test (FIG. 12). The T75 recovery time in PLR 128 achieved a mean AUC of 0.706 that was previously shown with p<0.001.

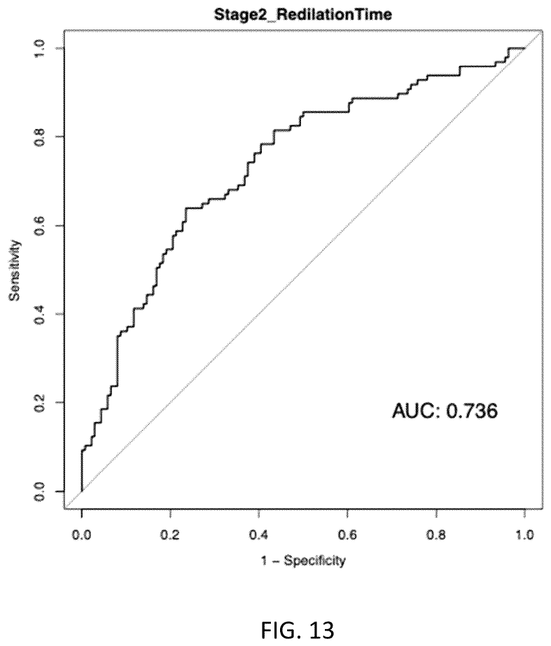

For PLR 255, the mean AUC of relative amplitude, percentage of pupil constriction and ⅓ redilation time were 0.732, 0.726 and 0.766 respectively (FIGS. 13A to 13C). All of the three parameters were demonstrated with statistical significance with p<0.001.

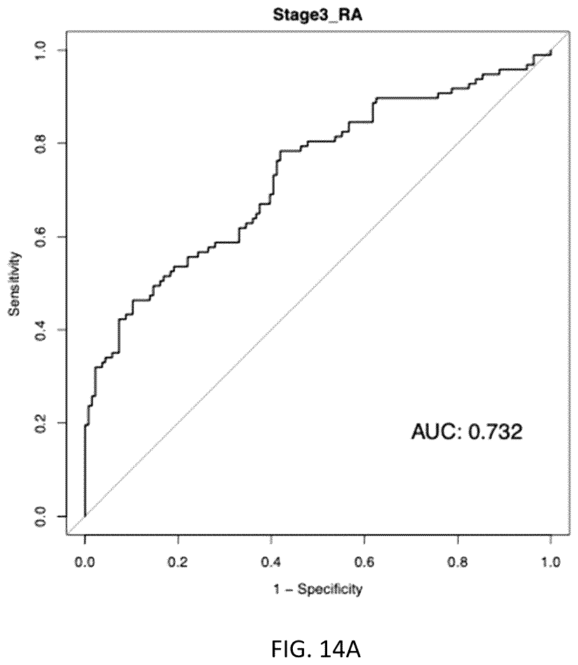

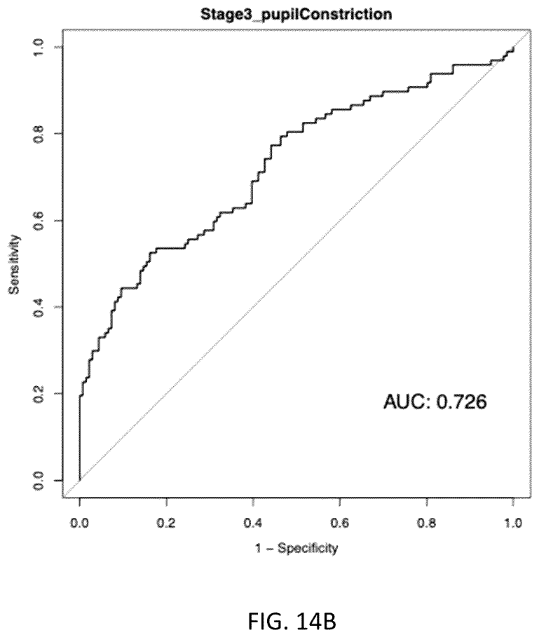

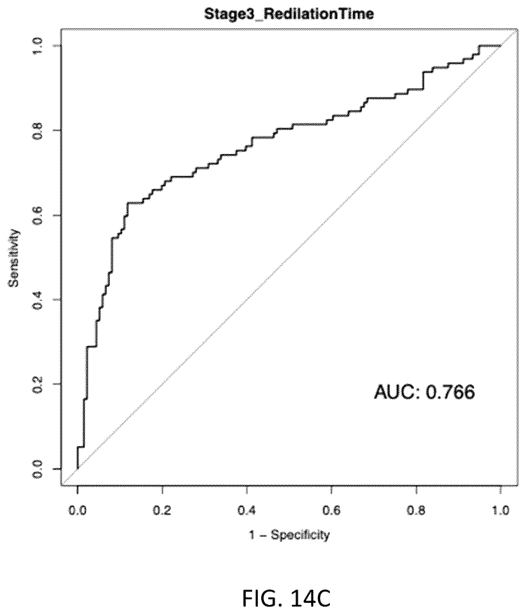

The dataset was split into 90% training sets and 10% testing sets randomly. A total of 10 pupillometric parameters were computed by the model, namely latency, relative amplitude, response time, % constriction time, average constriction velocity, percentage of pupil constriction, dilation velocity (rapid phase), dilation velocity (slow phase), ⅓ redilation time, and T75 recovery time. The model performance was quantified as Area Under the Receiver Operating Characteristic Curve (AUC), which represents the capability to distinguish between classes. It demonstrated that the mean AUC of the CNN-LSTM machine learning network was 0.794 (FIG. 14).

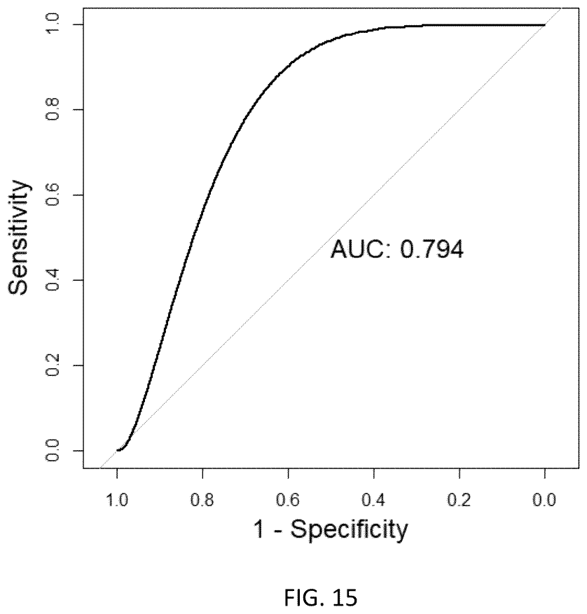

The difference between a pupillary unrest in the PSA group and a typical PLR in the HC group was also computed by the CNN-LSTM machine learning network. 90% of the data containing typical pattern of PLR and pupillary unrest was used as training sets while the remaining 10% was the testing samples. The mean AUC of the CNN-LSTM was 0.671 (FIG. 15).

### Example 3

Pupillary Light Responses (PLR) were recorded in response to 3 intensities of light stimuli (L6, L7 and L8) using a virtual reality head-mount display (VR-HMD). 9 PLR waveform features for each stimulus, were extracted by 2 masked observers and statistically analyzed. The inventors also used various methods on the whole PLR waveform including trained, validated and tested (6:3:1) by machine learning models including Multi-layer Perceptron (MLP), Support Vector Machine (SVM), K-nearest Neighbors (KNN), Logistic Regression, Decision Tree, Random Forest and Long Short-Term Memory (LSTM) models for two and three-class classification into long-COVID (LCVD), post-COVID (PCVD) or control. Accuracies/AUC of individual or combination of PLR features and ML models using PLR features or whole pupillometric waveform.

In this experiment, PLR from a total 185 subjects including 112 LCVD, 44 PCVD and 29 age/sex-matched controls were analysed. Models examined the independent effects of age and sex. Constriction Time (CT) after the brightest stimulus (L8) is significantly associated with LCVD status (two-way ANOVA, false discovery rate (FDR)<0.001; multinominal logistic regression, FDR<0.05). The overall accuracy/AUC of CT-L8 alone in differentiating LCVD from control or from PCVD were 0.7808/0.8711 and 0.8654/0.8140 respectively. Using cross-validated backward stepwise variable selection, CT-L8, CT-L6, Constriction Velocity (CV)-L6 were most useful to detect LCVD while CV-L8 for PCVD from other groups. The accuracy/AUC of selected features were 0.8000/0.9000 (control versus LCVD) and 0.9062/0.9710 (PCVD versus LCVD), better than when all pupillometric features under different stimulus conditions were combined. The LSTM model analyzing whole pupillometric waveform achieved the highest accuracy/AUC at 0.9375/1.000 in differentiating LCVD from PCVD and a slightly lower accuracy of 0.7838 for three-class classification (LCVD-PCVD-control).

In this example, the system 100 was used to identify individuals of different groups or categories, namely: “Post-COVID”, “Long-COVID” and “Healthy Control”, in which: Post-COVID (PCVD) includes patients who were tested positive for SARCoV-2 by Polymerase Chain Reaction (PCR) or rapid-antigen test (RAT) but did not report any systematic symptom; Long-COVID (LCVD) includes patients who were tested positive for SARCoV-2 by PCR or RAT and experienced any one of the long-COVID symptoms for 12 weeks; and Healthy Control (CONTROL) includes individuals who were never tested positive of SARCoV-2 by PCR or RAT up to the day of when the experiment was conducted.

PLR was measured using built-in infrared pupillometry from a VR-HMD, HTC VIVE Cosmos (High Tech Computer (HTC) Corporation) in the same quiet, undisturbed room. After putting on the VR-HMD comfortably, each participant was first dark-adapted for 12 seconds, then sequentially exposed to three increasing intensities of light stimuli as RGB (64,64,64), RGB (128, 128, 128) and RGB (256, 256, 256), which are approximately 563.71 Td, 1127.79 Td and 2246.59 Td respectively, each lasted for 2 seconds, delivered through the built-in display. Subjects were asked to close both eyes for 5 seconds before the test and to blink naturally throughout the test.

The PLR waveforms obtained with age range by decades (20-30, 31-40, 41-50, 51-60) and COVID status (LCVD, PCVD, CONTROL) were processed through two-way ANOVA tests and multinomial logistic regression. Alternatively, extracted pupillometric features may be used as input for being processed by the machine learning processing engine. The following tables shows 9 parameters of Pupillary Light Reflex (PLR) being processed by the processing module.

| | Initials | |
|---|---|---|
| Name | (Unit) | Description |
| Baseline Pupil | BPD (mm) | Initial pupil diameter in a dark |
| Diameter | | environment. |
| Minimum | MD (mm) | The minimum pupil diameter |
| Diameter | | during each stimulus. |
| Absolut | ACA (mm) | Difference between the |
| Construction | | initial diameter and the |
| Amplitude | | smallest diameter. |
| Constriction | CV (mm/s) | Constriction movement speed |
| Velocity | | in a given period of time. |
| Constriction | LAT (ms) | Time required for the effective |
| Latency | | start of constriction after the |
| | | start of stimulation. |
| Constriction | CT (ms) | Time required for the pupil to |
| Time | | reach the peak of constriction. |
| Redilation | RV (mm/s) | Redilation speed after the end |
| Velocity | | of light stimulation. |
| PIPR | PIPR (%) | Pupil size at 10 s after each |
| Amplitude | | stimulus. |
| Time to | TR (ms) | Time required for the pupil to |
| Redilation | | return to a certain percentage |
| | | of the baseline (50%). |

Based on the results of association tests, boxplots of significant features were constructed to classify the three classes (LCVD, PCVD, CONTROL) and conducted cross-validated, backward stepwise variable selection for the logistic regression models in order to identify the most useful features to identify LCVD status. The cross validation process employed bootstrapping with sampling replacement to generate multiple resampled datasets from each original dataset, and selected optimal models based on the Akaike Information Criterion (AIC) using the 5%, 10% and 15% models with the lowest AICs.

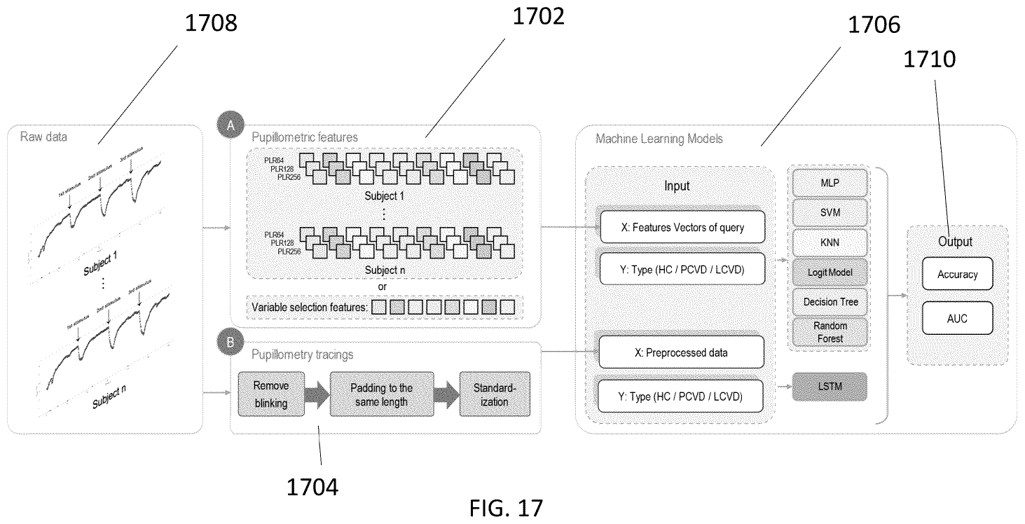

Finally, the inventors compared the performance of different machine learning models in differentiating the three classes (LCVD, PCVD, CONTROL) using the extracted PLR features and the whole pupillometry waveforms. The performance for two-class (CONTROL vs PCVD, CONTROL vs LCVD, PCVD vs LCVD) or three-class classification (LCVD vs PCVD vs CONTROL) are reported, and the data analysis workflow is shown in FIG. 17.

The first path, shown in box “A” 1702 in FIG. 17, involved manually extracting the 9 features from each of the PLR curve 1708 generated after the 3 light stimuli providing a total of 27 vectors. The inventors then used these features to create a feature vector (X) as the input for the machine learning models using the COVID status (LCVD, PCVD or CONTROL) as the outcome classifier. The machine learning models 1706 include Multi-layer Perceptron, Support Vector Machine, K-nearest Neighbors, Logistic Regression, Decision Tree and Random Forest.

The second path, shown in box “B” 1704 in FIG. 17, used data preprocessing to standardize the pupillometry waveform 1708 before inputting them as time series into the Long Short-Term Memory (LSTM) model. This preprocessing included removing pairs of blinking, padding sequences to achieve uniform length, and standardized the pupillometry waveform data. The preprocessed data were then used as the feature vectors (X) and the COVID status (LCVD, PCVD or CONTROL) were again used as the outcome (Y) input for the LSTM model in the module 1706.

The accuracy of the LSTM model in classification were also evaluated. The performance of the feature-based (A) and the raw data (B) approach in both two-class and three-class classification were compared using accuracy as an output 1710 to measure the overall correctness of the predictions made by each model. Additionally, AUC value as another output 1710 was calculated to illustrate the performance of the two-class classifiers.

Constriction Time (CT) was found particularly useful by different statistical methods. The best accuracy of differentiating CONTROL from LCVD and PCVD from LCVD was 0.8889 and 0.9333 respectively, both after the strongest light stimuli (L8) in subjects aged 31-40 using the cut-off value 0.6648 second (CONTROL from LCVD) and 0.7766 second (PCVD from LCVD) respectively. When considering subjects in all age ranges, the CT-L8 remained the most accurate feature. In summary, the CT-L8 alone achieved good accuracy to differentiate CONTROL from LCVD at 0.7808 and PCVD from LCVD at 0.8654. In addition, the experimental results also showed that Constriction Velocity, in particular (CV)-L6, was useful to detect LCVD.

These embodiments may be advantageous in that, the system allow long-COVID or post COVID detection and monitoring using pupillometry. Improvement in following conditions or disease detection: long-COVID and post-COVID: This non-intrusive, efficient system and method can be used for detecting long-COVID and post-COVID conditions.

Advantageously, the system may provide a solution which overcomes challenges in detecting long-COVID is identifying its neurological signs, which can be subtle and difficult to diagnose. The system is provided to capture, identify, and characterize pupillometry tracings. These tracings provide valuable information about the pupil reactivity and response, which facilitate classifying individuals into different types, namely Healthy Control (HC), Post-COVID (PCVD), and long-COVID (LCVD). The practical use of this invention is to aid in the detection, classification and monitor of individuals with long-COVID and post-COVID condition.

Advantageously, by comparing the determined parameters, such as Baseline Pupil Diameter, Minimum Diameter, Absolute Construction Amplitude, Constriction Velocity, Constriction Latency, Construction Time, Redilation Velocity, and post-illumination pupil response (PIPR) Amplitude, with a corresponding control parameter, symptoms and signs associated with particular health conditions may be identified. This enables healthcare professionals or patient itself to home monitor and provide appropriate medical interventions and treatment plans for individuals experiencing prolonged effects or complications from COVID19. Additionally, this system and method can contribute to a better understanding of long-COVID and its neurological manifestations, leading to improved management and care for affected individuals.

Furthermore, this invention also includes a non-intrusive system and method for detecting pupil reactivity and pupil response of a subject for the detection of psychoactive substance abuse (PSA). The method involves tracking eye pupil size, pupil reactivity, and pupil response of the subject, identifying and characterizing the dynamics of the pupil tracing to determine one or more parameters. The aim of this invention is to aid law enforcement agencies, drug rehabilitation centers, schools, and other relevant organizations in the early detection and classification of individuals under the influence of psychoactive substances. By providing a nonlabelling, non-invasive, and portable solution for drug detection, this system and method can contribute to more effective drug monitoring and intervention strategies, ultimately promoting public safety and health.

For the respective of detecting long-COVID, while there may be devices and diagnostic tools available that can assess certain symptoms or complications of COVID-19, for example elevating heart rate, heart rate variability (HRV), resting heart rate (RHR), sleeping duration, but none specifically focus on the neurological manifestations associated with long-COVID.

Other diagnostic methods often involve invasive procedures (Blood taking), expansive method, like MRI or rely on subjective assessments (questionnaire), which may not accurately and affordably capture the subtle neurological signs and variations seen in long-COVID.

### Embodiments in Accordance with the Present Invention Also Provide the Following Advantageous

1. Binocular Capability: Unlike current methods that only capture data from one eye, this invention allows for binocular pupillometry. By capturing and analyzing data from both eyes simultaneously, a more precise evaluation of a subject's pupillometry response can be generated. This enables more accurate and robust assessments of the different conditions.

2. Both Capture and Analysis: In contrast to current approaches that solely focus on capturing pupillometry tracing, this invention goes beyond and includes detailed analysis of the captured data. By characterizing the dynamics of the pupil tracing and determining multiple parameters in different conditions, such as baseline pupil diameter, constriction velocity, redilation velocity, and more, the method provides a deeper level of analysis for precise evaluation.

3. Software Upgradability: A key advantage of this invention is its ability to be easily upgraded via software updates. The system can be programmed to emit different light waves, vary light duration, present specific video stimuli, or display patterned images. This flexibility allows for the detection of various conditions by adapting to specific diagnostic requirements. As new research or diagnostic criteria emerge, the system can be updated accordingly without requiring hardware modifications.

Advantageously, by offering a non-intrusive solution that can effectively capture and analyze pupillometry tracings, this invention provides a significant advancement in the field of long-COVID as well as psychoactive drug detection.

In addition, regarding PSA detection, for the respective of detecting psychoactive drugs abusers, existing methods for detecting psychoactive drugs typically involve invasive or inefficient procedures such as urine or blood tests. These methods may also require specialized equipment or lab-testing facilities, making them inconvenient and time-consuming for law enforcement agencies, drug rehabilitation centers, schools, and other relevant organizations.

In contrast, this invention introduces a novel approach to identifying and classifying individuals based on pupillometry tracings and specific parameters. The non-intrusive and efficient IP&ET system and method offer several advantages over existing products:

1. Non-invasive: The system does not require any invasive procedures such as blood or urine collection. Instead, it utilizes pupillometry and eye-tracking, which is a non-intrusive method that can quickly and easily measure pupil reactivity and response.

2. Non-labelling: Unlike traditional drug detection methods that involve labeling individuals as drug users, the system and method do not stigmatize or label individuals. By using non-invasive pupillometry analysis, the system provides a discreet and non-judgmental approach to drug detection. This non-labeling aspect can encourage more individuals to voluntarily participate in testing and seek necessary support or intervention if needed, thereby promoting a more inclusive and compassionate approach to addressing substance abuse issues.

3. Binocular pupilometry: the invention revolutionizes pupilometry by introducing a cutting-edge binocular method that captures pupilometry responses from both eyes simultaneously. In contrast to the traditional monocular devices that only measure a single eye's pupil response, binocular pupilometry method offers a significant advantage. Users and professionals can now obtain synchronized and accurate data from both eyes, enabling more precise analysis and unlocking a new level of insight into physiological and psychological conditions that manifest through the eyes.

4. Accuracy and specificity: The system enable the selective capture and comparison of key pupillometry parameters for Long-COVID and psychoactive drug detection. By comparing these parameters with corresponding control parameters, the system can accurately distinguish between individuals who are under the influence of Long-COVID or psychoactive substances and those who are not.

5. Early detection and home monitor: The system aims to aid in the early detection and classification of individuals under the influence of neurological manifestations or psychoactive substances. This early detection and home care can allow for timely intervention and appropriate support, contributing to better outcomes for individuals and ultimately promoting public safety and health.

Eye Although not required, the embodiments described with reference to the Figures can be implemented as an application programming interface (API) or as a series of libraries for use by a developer or can be included within another software application, such as a terminal or personal computer operating system or a portable computing device operating system. Generally, as program modules include routines, programs, objects, components, and data files assisting in the performance of specific functions, the skilled person will understand that the functionality of the software application may be distributed across a number of routines, objects, or components to achieve the same functionality desired herein.

It will also be appreciated that where the methods and systems of the present invention are either wholly implemented by computing systems or partly implemented by computing systems then any appropriate computing system architecture may be utilized. This will include stand-alone computers, network computers and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. A method for determining a health condition of a subject, comprising the steps of:
providing visual stimulus to the subject;
recording a binocular response performed by the subject in response to the visual stimulus being received; and
analyzing a plurality of parameters extracted from the recorded binocular response to determine the neurological status and/or neurological activity of the subject, thereby determining one or more symptoms associated with the health condition of the subject.

2. The method in accordance with claim 1, wherein the binocular response includes binocular pupillometry response and reactivity.

3. The method in accordance with claim 2, wherein the plurality of parameters includes at least one of maximum pupil diameter, minimum pupil diameter, absolute construction amplitude, constriction velocity, constriction latency, construction time, redilation velocity and post-illumination pupil response amplitude, relative amplitude, ⅓ redilation time, percentage of pupil constriction, recovery time to achieve 75% of maximal pupil size (T75) and ⅔ constriction time, latency, response time, rapid phase dilation velocity and slow phase dilation velocity.

4. The method in accordance with claim 1, wherein the health condition includes disease or disorder caused by neurodevelopmental, neurodegenerative, neuropsychiatric, neurocognitive or neurobehavioral issues.

5. The method in accordance with claim 4, wherein the health condition includes long-COVID, post-COVID or psychoactive drug abuse.

6. The method in accordance with claim 2, wherein the binocular response further includes tracked eye movements in response to the visual stimulus.

7. The method in accordance with claim 1, wherein the visual stimulus is provided to the subject according to a stimulus protocol within a predetermined period of time.

8. The method in accordance with claim 7, further comprising the step of generating pupillary light reflex (PLR) data for each eye synchronized with the stimulus protocol based on the recorded binocular response, wherein the plurality of parameters are extracted from the pupillary light reflex data.

9. The method in accordance with claim 7, wherein the visual stimulus is provided to the subject using a VR head-mount display apparatus.

10. A system for determining a health condition of a subject, comprising:
a display module arranged to provide visual stimulus to the subject;
an image capturing module arranged to record a binocular response performed by the subject in response to the visual stimulus being received; and
a processor module arranged to analyze a plurality of parameters extracted from the recorded binocular response to determine the neurological status and/or neurological activity of the subject, thereby determining one or more symptoms associated with the health condition of the subject.

11. The system in accordance with claim 10, wherein the binocular response includes binocular pupillometry response and reactivity.

12. The system in accordance with claim 11, wherein the plurality of parameters includes at least one of maximum pupil diameter, minimum pupil diameter, absolute construction amplitude, constriction velocity, constriction latency, construction time, redilation velocity and post-illumination pupil response amplitude, relative amplitude, ⅓ redilation time, percentage of pupil constriction, recovery time to achieve 75% of maximal pupil size (T75) and ⅔ constriction time, latency, response time, rapid phase dilation velocity and slow phase dilation velocity.

13. The system in accordance with claim 10, wherein the health condition includes disease or disorder caused by neurodevelopmental, neurodegenerative, neuropsychiatric, neurocognitive or neurobehavioral issues.

14. The system in accordance with claim 13, wherein the health condition includes long-COVID or post-COVID or psychoactive drug abuse.

15. The system in accordance with claim 11, wherein the binocular response further includes eye movements in response to the visual stimulus.

16. The system in accordance with claim 10, wherein the display module is arranged to provide visual stimulus associated with a stimulus protocol within a predetermined period of time.

17. The system in accordance with claim 16, wherein the stimulus protocol is programmable, and wherein the stimulus protocol includes attributes including at least one of light intensity, duration, interval, wavelength, specific video stimuli and displayed pattern/images.

18. The system in accordance with claim 16, wherein the processor module is arranged to generate pupillary light reflex (PLR) data synchronized with the stimulus protocol for each eye based on the recorded binocular response, wherein the plurality of parameters are extracted from the pupillary light reflex data.

19. The system in accordance with claim 18, wherein the processor module includes a machine learning processing network.

20. The system in accordance with claim 16, wherein the display module includes a VR head-mount display apparatus comprising an eye spacing adjustment mechanism for adjusting a distance between a display of the VR head-mount display apparatus and eyes of the subject.
